(19) United States
(12) Patent Application Publication
(10) Pub. No.: US 2021/0259572 A1
(43) Pub. Date: Aug. 26, 2021

(54) IMAGING METHOD

(71) Applicant: Navix International Limited, Road Town, Tortola (VG)

(72) Inventor: Shlomo BEN-HAIM, Milan (IT)

(73) Assignee: Navix International Limited, Road Town, Tortola (VG)

(21) Appl. No.: 17/256,692

(22) PCT Filed: Jul. 4, 2019
(86) PCT No.: PCT/IB2019/055731

§ 371 Date: Dec. 29, 2020

Related U.S. Application Data
(60) Provisional application No. 62/693,930, filed on Jul. 4, 2018.

(51) Int. Cl. A61B 5/0536; A61B 5/0538; A61B 5/00; G06T 11/00

(52) U.S. Cl. CPC A61B 5/0536; A61B 5/0538; A61B 2576/023; G06T 11/006; A61B 5/0044

(57) ABSTRACT

A tissue imaging method of imaging a target tissue including: providing a plurality of hardware electrodes; sending and receiving electrical signals using the plurality of hardware electrodes to produce measurement data; selecting a virtual electrode model, where each the virtual electrode includes two or more hardware electrodes; processing the measurement data using the virtual electrode model to provide a processed data output; and reconstructing an image using the processed data output.

### RELATED APPLICATIONS

This application claims the benefit of priority under 35 USC § 119(e) of U.S. Provisional Patent Application No. 62/693,930 filed Jul. 4, 2018; the contents of which are incorporated herein by reference in their entirety.

### FIELD AND BACKGROUND OF THE INVENTION

The present invention, in some embodiments thereof, relates methods and tools for tissue imaging and, more particularly, but not exclusively, to methods of and tools for imaging of tissue using electrical sensing.

Electrodes inserted within the body (intrabody electrodes)—for example, electrodes on electrode catheters sized for vascular insertion—may be functionally connected to electrical field generating and/or measurement equipment enabling use of the electrodes as part of a measurement system. Measurements may be used to determine, for example, electrode position and/or to map tissue and/or tissue properties.

Among the electrical parameters which may be measured is impedance; that is, impedance of tissue affecting an electrical field which the intrabody electrodes generate and/or sense.

### SUMMARY OF THE INVENTION

There is provided, in accordance with some embodiments of the present disclosure, a tissue structure imaging method comprising: receiving a first set and a second set of electrical field measurement data measured by respective first and second in-body electrodes from positions on respective first and second sides of the tissue structure; and generating an image of the tissue structure using the first and second sets of electrical field measurement data; wherein the positions on the first side and the second side are spatial locations with the tissue structure between them, and wherein there is, for each spatial location, a surface of the tissue exposed to it across a fluid medium, and the exposed surfaces each have a non-overlapping portion comprising at least 20% of their surface.

In some embodiments, the generating the image comprises transforming measurements to locations under a constraint that the two sets of electrical field measurements are of different sides of the same tissue structure.

In some embodiments, the exposed surfaces are on lines of sight from each of the positions on the first side and the second side which meet at 180°.

In some embodiments, the measurements collected from both sides of the tissue structure comprise measurements indicative of currents applied to the first electrode and voltage measurements by the second electrode; and measurements indicative of currents applied to the second electrode and voltage measurements by the first electrode.

In some embodiments, the generating comprises solving the inverse problem to produce an image of the tissue structure using the measurement data, including comparing differences in measurement data obtained from the positions on the first and second sides to constrain the solution of the inverse problem.

In some embodiments, a respective straight path from each of the first and second electrodes to the tissue structure passes through a fluid medium without the straight path penetrating through or into another solid structure, and wherein the generating comprises using this positioning as a constraint on the solution of the inverse problem.

In some embodiments, the generating the image comprises generating a respective first location cloud and second location cloud for locations of portions of the tissue structure using the first and second sets of electrical field measurement data, and then combining the first location cloud and second location cloud to generate the image.

In some embodiments, the combining comprises averaging locations of corresponding features within the first and second location clouds.

In some embodiments, the combining comprises discarding at least one feature present in one of the first location cloud and the second location cloud, but not shown in the other.

In some embodiments, each line of sight defines a straight path including the spatial location and the tissue structure, and passing through a medium without the straight path penetrating through or into another solid structure.

In some embodiments, the method comprises: positioning the first in-body electrode on a first side of the tissue structure and the second in-body electrode on a second side of the tissue structure; and using the electrodes to collect measurement data of one or more electrical fields passing through the tissue structure, the measurement data comprising the first set and the second set of electrical field measurement data.

There is provided, in accordance with some embodiments of the present disclosure, the tissue structure imaging method described above, comprising positioning a third electrode on a third side of the tissue structure.

In some embodiments, the first electrode and the second electrode are both part of a same imaging tool.

There is provided, in accordance with some embodiments of the present disclosure, the tissue structure imaging method described above, wherein the positioning includes positioning a first set of electrodes on the first side and a second set of electrodes on the second side.

There is provided, in accordance with some embodiments of the present disclosure, the tissue structure imaging method described above, wherein the first set of electrodes and the second set of electrodes are both parts of a same imaging tool.

There is provided, in accordance with some embodiments of the present disclosure, the tissue structure imaging method described above, wherein the positioning comprises inserting a portion of a tool with the first electrode through the tissue structure.

There is provided, in accordance with some embodiments of the present disclosure, the tissue structure imaging method described above, wherein the tissue structure is a vascular system valve.

There is provided, in accordance with some embodiments of the present disclosure, the tissue structure imaging method described above, wherein the tissue structure is a heart valve.

There is provided, in accordance with some embodiments of the present disclosure, the tissue structure imaging method described above, comprising using the image to guiding placement of a device with respect to the tissue structure.

There is provided, in accordance with some embodiments of the present disclosure, the tissue structure imaging method described above, wherein the tissue structure is a cardiovascular valve and the device is a valve clip.

There is provided, in accordance with some embodiments of the present disclosure, the tissue structure imaging method described above, wherein the valve clip comprises one or more of the electrodes.

There is provided, in accordance with some embodiments of the present disclosure, the tissue structure imaging method described above, wherein a delivery device for the valve clip comprises one or more of the electrodes.

There is provided, in accordance with some embodiments of the present disclosure, the tissue structure imaging method described above, comprising generating an electrical field at a volume including a volume of the tissue structure; wherein the measurement data comprises features of the electrical field affected by the tissue structure.

There is provided, in accordance with some embodiments of the present disclosure, the tissue structure imaging method described above, wherein the generating an electrical field comprises generating at least three electromagnetic fields, where the fields have crossing orientations.

There is provided, in accordance with some embodiments of the present disclosure, the tissue structure imaging method described above, wherein the fields have different frequencies.

There is provided, in accordance with some embodiments of the present disclosure, a tissue structure imaging method comprising: receiving a first set and a second set of electrical field measurement data measured by respective first and second in-body electrodes from positions on respective first and second sides of the tissue structure; and generating an image of the tissue structure using the first and second sets of electrical field measurement data; wherein the tissue structure is a cardiovascular valve, and the positions on the first side and the second side are each a spatial location within a cardiovascular lumen on a different side of the cardiovascular valve.

In some embodiments, the generating the image comprises transforming measurements to locations under a constraint that the two sets of electrical field measurements are of different sides of the same tissue structure.

In some embodiments, the measurements collected from both sides of the tissue structure comprise measurements indicative of currents applied to the first electrode and voltage measurements by the second electrode; and measurements indicative of currents applied to the second electrode and voltage measurements by the first electrode.

In some embodiments, the generating comprises solving the inverse problem to produce an image of the tissue structure using the measurement data, including comparing differences in measurement data obtained from the positions on the first and second sides to constrain the solution of the inverse problem.

In some embodiments, a respective straight path from each of the first and second electrodes to the tissue structure passes through a fluid medium without the straight path penetrating through or into another solid structure, and wherein the generating comprises using this positioning as a constraint on the solution of the inverse problem.

There is provided, in accordance with some embodiments of the present disclosure, the tissue structure imaging method described above, comprising using the image to guide placement of a device with respect to the tissue structure.

There is provided, in accordance with some embodiments of the present disclosure, the tissue structure imaging method described above, comprising using the image to guide attachment of a device to the tissue structure.

In some embodiments, the method comprises: positioning the first in-body electrode on a first side of the tissue structure and the second in-body electrode on a second side of the tissue structure; and using the electrodes to collect measurement data of one or more electrical fields passing through the tissue structure, the measurement data comprising the first set and the second set of electrical field measurement data.

There is provided, in accordance with some embodiments of the present disclosure, a tissue structure imaging method comprising: receiving a first set and a second set of electrical field measurement data measured by respective first and second in-body electrodes from positions on respective first and second sides of the tissue structure; and generating an image of the tissue structure using the first and second sets of electrical field measurement data; and using the image to guide attachment of a device to the tissue structure.

In some embodiments, the generating the image comprises transforming measurements to locations under a constraint that the two sets of electrical field measurements are of different sides of the same tissue structure.

In some embodiments, the measurements collected from both sides of the tissue structure comprise measurements indicative of currents applied to the first electrode and voltage measurements by the second electrode; and measurements indicative of currents applied to the second electrode and voltage measurements by the first electrode.

In some embodiments, the generating comprises solving the inverse problem to produce an image of the tissue structure using the measurement data, including comparing differences in measurement data obtained from the positions on the first and second sides to constrain the solution of the inverse problem.

In some embodiments, a respective straight path from each of the first and second electrodes to the tissue structure passes through a fluid medium without the straight path penetrating through or into another solid structure, and wherein the generating comprises using this positioning as a constraint on the solution of the inverse problem.

In some embodiments, the method comprises: positioning the first in-body electrode on a first side of the tissue structure and the second in-body electrode on a second side of the tissue structure; and using the electrodes to collect measurement data of one or more electrical fields passing through the tissue structure, the measurement data comprising the first set and the second set of electrical field measurement data.

There is provided, in accordance with some embodiments of the present disclosure, a tissue structure imaging method comprising: receiving a first set and a second set of electrical field measurement data measured by respective first and second in-body electrodes from positions on respective first and second sides of the tissue structure; and generating an image of the tissue structure using the first and second sets of electrical field measurement data; an wherein generating the image comprises determining a solution to the inverse problem, and the determining a solution to the inverse problem uses measurements collected from both sides of the tissue structure.

In some embodiments, the measurements collected from both sides of the tissue structure comprise measurements indicative of currents applied to the first electrode and voltage measurements by the second electrode; and measurements indicative of currents applied to the second electrode and voltage measurements by the first electrode.

In some embodiments, the generating the image comprises transforming measurements to locations under a constraint that the two sets of electrical field measurements are of different sides of the same tissue structure.

In some embodiments, the generating comprises solving the inverse problem to produce an image of the tissue structure using the measurement data, including comparing differences in measurement data obtained from the positions on the first and second sides to constrain the solution of the inverse problem.

In some embodiments, a respective straight path from each of the first and second electrodes to the tissue structure passes through a fluid medium without the straight path penetrating through or into another solid structure, and wherein the generating comprises using this positioning as a constraint on the solution of the inverse problem.

In some embodiments, the method comprises: positioning the first in-body electrode on a first side of the tissue structure and the second in-body electrode on a second side of the tissue structure; and using the electrodes to collect measurement data of one or more electrical fields passing through the tissue structure, the measurement data comprising the first set and the second set of electrical field measurement data.

There is provided, in accordance with some embodiments of the present disclosure, a system for tissue structure imaging, comprising: a first electrode and a second electrode configured to be placed in intrabody locations, each on a different side of an internal tissue structure to be imaged; an electrical field signal generator/measurer, functionally connected to the first and second electrodes to send and receive electrical signals; a control unit, configured to solve the inverse problem to produce an image of the internal tissue structure using measurements received as electrical signals by the electrical field signal generator/measurer, including comparing differences in measurement data obtained from the different sides of the internal tissue structure to constrain the solution of the inverse problem.

In some embodiments, the measurements comprise measurements indicative of currents applied to the first electrode and voltage measurements by the second electrode; and measurements indicative of currents applied to the second electrode and voltage measurements by the first electrode.

In some embodiments, a respective straight path from each of the first and second electrodes to the internal tissue structure passes through a fluid medium without the straight path penetrating through or into another solid structure, and wherein the control unit is configured to use this positioning as a constraint on the solution of the inverse problem.

In some embodiments, the solution of the inverse problem comprises transforming measurements to locations under a constraint that the two sets of electrical field measurements are of different sides of the same tissue structure.

In some embodiments, control unit is configured to send and receive electrical signals to obtain the measurements from both sides of the tissue structure, including measurements indicative of currents applied to the first electrode and voltage measurements by the second electrode; and measurements indicative of currents applied to the second electrode and voltage measurements by the first electrode.

There is provided, in accordance with some embodiments of the present disclosure, a tissue imaging method of imaging a target tissue comprising: providing a plurality of hardware electrodes; sending and receiving electrical signals using the plurality of hardware electrodes to produce measurement data; selecting a virtual electrode model, where each virtual electrode includes two or more hardware electrodes; processing the measurement data using the virtual electrode model to provide a processed data output; and reconstructing an image using the processed data output.

There is provided, in accordance with some embodiments of the present disclosure, the tissue imaging method described above, wherein the sending and receiving is simultaneous.

There is provided, in accordance with some embodiments of the present disclosure, the tissue imaging method described above, wherein the sending and receiving comprises sending and receiving of signals through and from the target tissue; wherein the reconstructing is of an image of the target tissue.

There is provided, in accordance with some embodiments of the present disclosure, the tissue imaging method described above, wherein the selecting a virtual electrode model is based on one or more of: a desired spatial resolution; a volumetric accuracy; a sensing location; and a spatial limitation of the target tissue.

There is provided, in accordance with some embodiments of the present disclosure, the tissue imaging method described above, wherein the processing comprises processing measurement data so that at least a subset of the plurality of hardware electrodes operates as a phased array.

Following are examples of some embodiments of the present disclosure. Features of one example may be combined with features of one or more other examples, unless expressly prohibited and form additional examples of some embodiments of the present disclosure.

Example 1. A tissue imaging method of imaging a target tissue comprising:

providing a plurality of hardware electrodes; sending and receiving electrical signals using said plurality of hardware electrodes to produce measurement data; selecting a virtual electrode model, where each said virtual electrode includes two or more hardware electrodes; and processing said measurement data using said virtual electrode model to provide a processed data output; reconstructing an image using said processed data output.

Example 2. The tissue imaging method according to Example 1, wherein said sending and receiving is simultaneous.

Example 3. The tissue imaging method according to any one of Examples 1-2, wherein said sending and receiving comprises sending and receiving of signals through and from said target tissue; wherein said reconstructing is of an image of the target tissue.

Example 4. The tissue imaging method according to any one of Examples 1-3, wherein said selecting a virtual electrode model is based on one or more of: a desired spatial resolution; a volumetric accuracy; a sensing location; and a spatial limitation of said target tissue.

Example 5. The tissue imaging method according to any one of Examples 1-4, wherein said processing comprises processing measurement data so that at least a subset of said plurality of hardware electrodes operates as a phased array.

Example 6. A tissue structure imaging method comprising: positioning a first electrode on a first side of the tissue structure and a second electrode on a second side of the tissue structure; sending and receiving electrical signals using said electrodes to collect measurement data; and generating an image of said tissue structure using said measurement data collected from both sides of the tissue structure.

Example 7. The tissue structure imaging method according to Example 6, wherein said first side and said second side are spatial locations surrounding the structure where there is a line of sight from the spatial location to the structure.

Example 8. The tissue structure imaging method according to any one of Examples 6-7, wherein said first side and said second side are separated, for one or more dimension, by at least 50% of a size of the first electrode in said dimension.

Example 9. The tissue structure imaging method according to any one of Examples 6-8, comprising positioning a third electrode on a third side of said structure.

Example 10. The tissue structure imaging method according to any one of Examples 6-9, wherein said positioning includes positioning said first electrode on said first side and said second electrodes on said second side.

Example 11. The tissue structure imaging method according to Example 10, wherein said first electrode and said second electrode are part of an imaging tool.

Example 12. The tissue structure imaging method according to any one of Examples 6-11, wherein said positioning includes positioning a first set of electrodes on said first side and a second set of electrodes on said second side.

Example 13. The tissue structure imaging method according to Example 12, wherein said first set of electrodes and said second set of electrodes are part of an imaging tool.

Example 14. The tissue structure imaging method according to any one of Examples 6-13, wherein said positioning comprises inserting portion of tool with first electrode through the structure.

Example 15. The tissue structure imaging method according to any one of Examples 6-14, wherein the structure is a vascular system valve.

Example 16. The tissue structure imaging method according to any one of Examples 6-15, wherein the structure is a heart valve.

Example 17. The tissue structure imaging method according to any one of Examples 6-16, comprising using said image to guiding placement of a device with respect to the structure.

Example 18. The tissue structure imaging method according to Example 17, wherein the structure is a vascular valve and said device is a valve clip.

Example 19. The tissue structure imaging method according to Example 18, wherein said valve clip comprises one or more of said electrodes.

Example 20. The tissue structure imaging method according to any one of Examples 18-19, wherein a delivery device for said valve clip comprises one or more of said electrodes.

Example 21. The tissue structure imaging method according to any one of Examples 5-20, comprising generating a field at a volume including a volume of the structure;

wherein said measurement data comprises changes to said field.

Example 22. The tissue structure imaging method according to Example 21, wherein said changes to said field are associated with said sending.

Example 23. The tissue structure imaging method according to any one of Examples 21-22, wherein said changes to said field are associated with said structure.

Example 24. The tissue structure imaging method according to any one of Examples 21-23, wherein said generating comprises generating at least three electromagnetic fields, where said fields have crossing orientations.

Example 25. The tissue structure imaging method according to Example 24, wherein said fields have different frequencies.

Example 26. A method of imaging a target tissue comprising: providing a plurality of hardware electrodes; sending and receiving electrical signals using said plurality of hardware electrodes to produce measurement data; selecting a virtual electrode by: selecting a subset of said hardware electrodes; selecting, to transform said measurement data into data provided by said virtual electrode by processing of one or more of: signals transmitted by said plurality of hardware electrodes; and signals received by said plurality of hardware electrodes; processing said measurement data using said virtual electrode.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable memory to receive instructions and/or data therefrom, process them, and/or store processing results in the same or another computer readable storage memory. The processing performed (optionally on the data) is specified by the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as collecting dental measurements, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1 is a simplified schematic of a system for imaging tissue within a patient, according to some embodiments of the invention.

FIG. 2 is a flow chart of a method of tissue imaging, according to some embodiments of the invention;

FIG. 3 is a simplified schematic of an imaging tool including a single electrode, according to some embodiments of the invention;

FIGS. 4-5 are simplified schematics of imaging tools including two electrodes, according to some embodiments of the invention;

FIGS. 6-9 are simplified schematics of imaging tools including a plurality of electrodes, according to some embodiments of the invention;

FIG. 10 is a flow chart of a method of measurement of a structure using electrodes positioned on at least two sides of the structure, according to some embodiments of the invention;

FIG. 11 is a simplified schematic of an imaging tool where a first set of electrodes are positioned on a first side of tissue to be imaged and a second set of electrodes are positioned on a second side of tissue to be imaged, according to some embodiments of the invention;

FIGS. 12A-B are simplified schematics cross sections showing placement of a valve clip assisted by an imaging tool, according to some embodiments of the invention;

FIG. 13 is a simplified schematic of a virtual electrode, according to some embodiments of the invention;

FIG. 14 is a simplified schematic of a virtual electrode, according to some embodiments of the invention;

FIG. 15 is a flow chart depicting a method of dielectric mapping, optionally for imaging a body volume or for reconstructing body volume, according to some embodiments of the present disclosure; and

FIG. 16 is a flowchart schematically representing a method of finding a solution to the inverse problem, according to some embodiments of the present disclosure;

### DESCRIPTION OF SPECIFIC EMBODIMENTS OF THE INVENTION

The present invention, in some embodiments thereof, relates methods and tools for tissue imaging and, more particularly, but not exclusively, to methods of and tools for imaging of tissue using electrical sensing.

### Overview

A broad aspect of some embodiments of the invention relates to measurement of tissue where one or more probe is used for electrical sensing where, in some embodiments, each probe includes one or more electrodes. In some embodiments, sensed measurements are used to construct image(s). In some embodiments, sensing includes near and/or medium field electrical measurements, from within a patient's body, wherein the at least one electrical field measured passes through the measured tissue. In some embodiments, electrode(s) are used in impedance spectroscopy e.g. dielectric spectroscopy.

In some embodiments, the method of measurement and image construction comprises generating a dielectric map of a region of an organ of a human or animal body using intrabody electrodes that were or are disposed inside or adjacent the region. In some embodiments, the intrabody electrodes are moved through the region collecting measurements. Electrical fields passing through the region (including through tissues of the region) interact with dielectric properties of the region (in particular, dielectric properties of tissues in the region). This in turn affects the distribution of electrical field potentials in the measurements, including influences on electrical field potentials measured away from (e.g., at least 1 cm, 2 cm, 3 cm, or another distance away from) the influencing regions themselves. Dielectric maps mapping different parts of the region, each part being mapped using the electrodes in a different position or orientation, are combined—for example, stitched together—to generate the dielectric map of the region. The dielectric map of each region provides a spatial distribution of one or more dielectric properties of tissue in the mapped region. The tissue may be, for example, blood, muscle, bone, nerve, and/or fat tissue. Examples of dielectric properties that may be mapped in the dielectric map include: conductivity, complex conductivity, real or imaginary part of conductivity, permittivity, complex permittivity, real or imaginary part of permittivity, and/or impedance.

In some embodiments, a method of generating a dielectric map of one or more dielectric properties in a region of an organ of a human or animal body comprises accessing a first plurality of data sets wherein each data set of the first plurality comprises measured voltage data indicative of voltages measured at a respective second set of one or more electrodes in response to electric fields in the region generated by currents applied to a respective first set of one or more electrodes.

Optionally, respective pairs of sets of electrodes are used in obtaining the first plurality of data sets; each pair comprising an electrode for generating field(s) in response to applied currents and an electrode for measuring voltages due to the generated fields.

First and second sets of electrodes (of the respective pairs) comprise electrodes disposed on a tool located at a first location in the region at the time of measurement. Herein, such a “tool” is a single instrument or instrument portion upon which both sets of electrodes are mounted, and comprising a single mechanically interconnected unit; e.g., the tool is actuable by control movements of a single handle, slider, or other mechanical device so that all parts of the single instrument portion move together. Examples of single tools include single catheters, scalpels, guide wires, sutures or other suitable surgical instruments. Pairings of such tools should be understood to constitute two separate tools.

The first and second sets of electrodes may have electrodes in common. Position data indicative of positions of the electrodes in the respective first and second sets of electrodes relative to the tool are also accessed. In this and in any other aspect of the disclosure, accessing position data and accessing a plurality of data sets can make part of a single step, or be carried out in different steps. The method further comprises generating at least a portion of the dielectric map by computing a first spatial distribution of one or more dielectric properties in the region using the first plurality of data sets and the position data.

In some embodiments, the method may further comprise:
determining the position of the tool in a reference frame fixed relative to the body, and
positioning the dielectric map in a reference frame fixed relative to the body based on the determined position.

Determining the position of the tool in a reference frame fixed relative to the body may comprise generating a global dielectric map of a portion of the body comprising the region, for example as described in the first aspect but with electrodes disposed in fixed relation to the body, for example, fixed to the skin of the patient or to a belt or garment worn by the patient, and determining the position of the tool based on the global dielectric.

In some embodiments, the method of measurement and image construction comprises use of Electrical Impedance Tomography (EIT). EIT systems and methods of medical imaging, as is known in the art, are implemented by deploying electrodes at the body's surface of a subject; injecting electrical excitation to some of the employed electrodes; measuring the electrical signals received at other employed electrodes; calculating, based on the measured signals, 3-D image(s) of tissues and organs inside the body; and providing a display of the calculated 3-D images. EIT techniques are based on the observation that muscle and blood conduct the applied currents better than fat, bone, or lung tissue and are therefore able to resolve different tissue types.

In some embodiments of the present disclosure, generating a dielectric map of a region of an organ of a human or animal body comprises use of constraint data representative of a distribution of dielectric properties of a tool that was or is disposed in or close to the region. The dielectric properties may be, for example, conductivity, complex conductivity, permittivity, complex permittivity and the like. The tool may be a catheter, scalpel, guide wire, suture or any suitable surgical instrument. The method then comprises computing the dielectric map as a spatial distribution of one or more dielectric properties in the region using the plurality of data sets, the position data, and the constraint data. In effect, the image produced is constrained to reproduce these properties. This method of introducing constraints potentially results in an improved EIT method, e.g., in terms of accuracy and/or precision of imaging of regions other than the tool which was used in developing the constraints.

Alternatively or additionally, constraint data may include constraint data as known in the art, for example: maximizing entropy in the obtained image. Still alternatively, the constraint data may include a parametric conductivity map, so that only the parameters are searched for.

The dielectric map provides a spatial distribution of one or more dielectric properties of tissue in the mapped region. The tissue may be, for example, blood, muscle, bone, nerve, and/or fat tissue. Examples of dielectric properties that may be mapped in the dielectric map includes, for example, conductivity, complex conductivity, real or imaginary part of conductivity, permittivity, complex permittivity, real or imaginary part of permittivity, and/or impedance.

In some embodiments, a method of generating a dielectric map of one or more dielectric properties in a region of an organ of a human or animal body comprises accessing a plurality of data sets acquired using respective pairs of sets (there being one or more electrodes in each set). For each pair of sets of electrodes, electric currents are applied to electrodes of one set of each pair (a respective first set) so as to generate electric fields in the region, and the other set of each pair (a respective second pair) is used to measure voltages generated in response to the application of the electric currents to the first set. Each dataset is based on measurements from a respective second set of electrodes. In some embodiments, the first and second sets of electrodes may have common electrodes. In some embodiments, each set of electrodes may be a respective single electrode.

Electrodes may be used to generate respective independent fields by exciting the respective fields (using the respective first sets of electrodes) in sequence and/or the respective independent fields may be generated by exciting some or all of the electrodes simultaneously but at different respective frequencies. In the latter case, the measurement at the corresponding second set of electrodes would be combined with signal processing to take measurements at the relevant frequency. For example, in some embodiments, a plurality of electrodes, possibly all but one available electrodes, each excite a field with a respective frequency and measurement of all these fields is done at the same ground electrode for all data sets. In this example, there is thus a data set for each of the plurality of electrodes, each having one of the plurality of electrodes constituting the first set of electrodes and the ground electrode constituting the second set of electrodes, with the electrodes disposed, for example as described below. Generally, in different data sets, the electrodes may be assigned to the first and second sets of electrodes in different ways, including inverting, between the two data sets, the roles of electrical current application and measurement of voltage for each electrode in the two sets of electrodes. Each data set thus represents an independent measurement and may include data acquired at different points in time and/or at different frequencies.

An aspect of some embodiments of the invention relates to imaging where sensing and/or transmitting is using virtual electrode(s). In some embodiments, each virtual electrode includes more than one hardware electrode, where the virtual electrode is mathematically reconstructed using data from transmitting and/or receiving electrodes.

In some embodiments, configuration of virtual electrodes is selected based on a desired spatial resolution and/or volumetric accuracy and/or shape and/or sensing location (e.g. with respect to a measurement volume) and/or angular and/or spatial limitation.

In some embodiments, virtual electrode(s) are formed by operating a plurality of electrodes as a phased array. In some embodiments, a phased array is operated (e.g. transmits and/or receives signals) at a single frequency where, in some embodiments, different phase changes are introduced to different hardware electrode received and/or transmitted signals.

In some embodiments, virtual electrode(s) are formed by processing of transmission and/or sensing data.

In some embodiments, sets of hardware electrodes (where, in some embodiments, a set includes at least two hardware electrodes) emit radiation simultaneously and/or sense simultaneously. A potential benefit of simultaneous emission and/or measurement is that a distance between the hardware electrodes) is constant during the emission and/or sensing.

In some embodiments, a virtual electrode is constructed by moving one or more hardware electrode in space while sensing using the hardware electrode(s). Where, in some embodiments, position of the electrode(s) being moved is estimated and/or sensed e.g. by electrodes which are not part of the moving virtual electrode. In some embodiments, a size of the virtual electrode is enlarged by moving hardware electrode(s). Where, once both measurements from hardware electrode(s) in the virtual electrode and position measurements of the virtual electrode hardware electrode(s) are collected, the data is reprocessed to provide measurements of a virtual electrode which is larger than the space taken up by the hardware electrode(s) of the virtual electrode. In some embodiments, a virtual electrode is constructed by processing of received signals from a plurality hardware electrodes. In some embodiments, a virtual electrode is constructed by controlling transmitted signal/s from a plurality of hardware electrodes.

In some embodiments, a signal is detected from each of a plurality of hardware electrodes. In some embodiments, a virtual electrode model is selected and the detected signals are then processed, using the model, to generate a signal that would have been received by a hardware electrode with the same characteristics as the virtual electrode model.

In some embodiments, phase-controlled signals are transmitted by each hardware electrode, the phase control defining characteristics of the virtual electrode.

An aspect of some embodiments of the invention relates to electrically sensing, using one or more probe (each probe including one or more electrode) on more than one side (e.g. 2 sides, 3 sides, 2-5 sides, or lower or higher or intermediate ranges or numbers of sides) of a structure to be measured (e.g. imaged). Herein, use of the term “structure” in relation to body organs and/or body tissue refers to non-fluidic structures, e.g., it excludes uncoagulated blood, but includes, for example, muscle, bone, connective tissue, secretory tissue, and/or neural tissue A structure optionally includes deposits such as plaque, and/or implanted devices. In some embodiments, a side of a structure is defined as a part of the structure bordering a region in which there is line of sight from each point in the region to each other point in the region (where the structure is the only object which can block the line of sight). Herein, a “line of sight” to a structure from a spatial location exists when the space intervening can be crossed by a straight path including the spatial location and the structure, and passing through a medium (that is, a fluid medium; e.g., air, blood and/or saline); without the path penetrating through or into another solid-phase structure such as solid body tissue. The term does not imply a particular restriction on the opacity or transparency of the fluid medium; nor does it entail “sight” in the visual sense. In particular, a line of sight, as the term is used herein, can extent through blood. Similarly, the extent of a surface of a structure which is “within a field of view” from a spatial location should be understood to be the surface seen as if viewed from that spatial location, disregarding obstruction by an intervening fluid medium such as blood. The surface within the field of view can also be characterized as the surface accumulating the end-points of all lines of sight. With reference to electrical measurements, the closest surface within the field of view in a certain direction offers the first discontinuity in dielectric properties in that direction. It is this type of discontinuity which modifies electrical fields in a manner that electrical field imaging uses to determine position clouds (and, in some embodiments, ultimately images) from measurements of the electrical field.

In some embodiments, lines of sight and/or relationships among fields of view from different sides of a tissue structure are used as constraints in solving the inverse problems.

In particular, an unobstructed (fluid being ignored) line of sight allows the simplifying assumption that starting from each electrode, the first structure encountered is also the structure which is to be imaged (that is, the electrical field is not influenced by an intervening non-fluid structure). Moreover, the first structure encountered is the same structure in both cases—a single structure must satisfy the further constraint of being consistent with data from both sides of the imaged structure. In the case of measuring a membranous structure such as a cardiovascular valve, there is potentially an even stronger constraint which may be applied, since the shape of the valve's membranous surface is substantially the same on either side. Furthermore, movements of the two sides of the structure are synchronized, and this synchronization may also be enforced on the solution using suitable constraints. The solution to the inverse problem optionally assumes membranous construction of the imaged structure as a constraint, in some embodiments of the present disclosure.

In some embodiments, different sides of a structure separate spatial locations where lines of sight from each spatial location to a near-side surface of the structure are lines of sight to at least partially (e.g., at least 20%, at least 40%, at least 60%, at least 80%, at least 90%, or optionally 100%) non-overlapping surface portions. In some embodiments, some sides of the structure are restricted by attached structures which “surround” the structure in the sense of blocking line of sight to a portion of the structure. For example, in the case of a blood vessel valve, a view of one side valve (e.g. the annulus of the valve at the blood vessel wall) is obstructed by the valve leaflets (where blood and/or fluid is not considered to obstruct the view). For example, in some embodiments, an annulus of a valve itself obstructs a view of the opposite side of the annulus.

Herein, the phrase “surrounding the structure” with reference to spatial locations from which measurements are taken should be understood to mean that the spatial locations are “positioned with the structure between them”. The “betweenness” holds for at least one pair of the spatial locations. Furthermore, “between” is understood to indicate a situation in which the structure has a respective surface portion exposed within a field of view to each of the spatial locations, and there is for each such respective surface portion within a field of view a sub-portion which is not part of the other surface portion within a field of view. This sub-portion is the “non-overlapping portion”; and that non-overlapping portion, in some embodiments, comprises at least 20% of the surface within a field of view, at least 40%, at least 60%, at least 80%, at least 90%, or 100% of the surface within a field of view from each of the spatial locations. In some embodiments, there exist respective lines of sight drawn from each location to the structure which meet at an angle of at least 145 degrees, 160 degrees; or at 180 degrees (that is, meet as a straight line). For purposes of determining whether two locations “surround” a structure with the structure between them, apertures of the structure (e.g., an aperture of a valve) are considered part of the structure, having a separate “surface” facing outward from either side of the aperture.

Additionally or alternatively, some embodiments, a first electrode (or first set of electrodes) is considered to be on a different side of the structure to a second electrode (or second set of electrodes) when, for one or more dimension, a distance between the electrodes (or set of electrodes) is at least 10%, at least 20%, at least 50%, at least 100% or 20%-300% of the size the first electrode in that dimension (or electrode set), and the structure is positioned between the two electrodes or electrode sets.

In some embodiments, sides of a structure are defined by a geometry of the structure. Where, in some embodiments, sides are portions of an external of the volume of the structure which oppose each other e.g. are opposite each other about a center of the structure.

A potential advantage of collecting measurements from electrodes located on more than one side of a structure, over, for example, collecting measurements from one side (e.g. using a camera), which, in the case of impedance imaging which can provide measurement of tissue surfaces and of tissue (e.g. tissue thickness) is increased accuracy. For example, as measurements of the surface of the side furthest from a single electrode (or group of electrodes) are not collected through a layer of tissue. This potentially allows simplifying a model or other method used in solving the inverse problem.

A further potential advantage of collecting measurements from electrodes located on more than one side of a structure (in particular when treating the structure e.g. attaching a valve clip to a vascular valve) is the ability to measure (e.g. image) both sides simultaneously. For example, when treating a structure where the treatment includes manipulating both sides of the structure. A 3-D model of the structure (e.g. generated using methods described in this document) is also potentially advantageous during treatment of the structure.

In some embodiments, both sides of the structure a measured using electrodes coupled to and/or attached to and/or part of a single imaging tool. A potential advantage being that if the tool is moved, image quality (e.g. of a 2-D image reconstructed using electrode measurement data), in some embodiments, is not affected, for example, moving the tool in one direction, in some embodiments, (e.g. where the tool includes a non-elastic connection between the two sets of electrodes) will increase a distance between the structure and one set of electrodes and decrease the distance to the structure of the other set of electrodes located at the other side of the structure.

In some embodiments, the structure comprises a valve, for example, a heart valve and/or a vascular valve (referred to herein as a valve of the cardiovascular system, or a cardiovascular valve). In an exemplary embodiment, measurement is of an annulus of a valve e.g. heart valve. As is well-known in the art, a cardiovascular valve occupies a portion of a cardiovascular lumen between two other cardiovascular lumen portions, and acts as a valve insofar as it acts to regulate fluid flow (e.g., restrict or prevent flow in a certain direction) between the two other cardiovascular portions. Herein, valves are used as examples of imaged structures, but it is to be understood that other cardiovascular structures may also be imaged using electrodes placed on either side of them. For example, in some embodiments the structure comprises a vascular stenosis, embolism or other vascular restriction, and/or malformation. In some embodiments, the structure comprises a region of vascular branching.

In some embodiments, one or more electrode is positioned in a first measurement region, on a first side of the structure (e.g. pre-valvularly) and one or more electrode is positioned in a second measurement region, on a second side of the structure (e.g. post-valvularly).

In some embodiments, data collected from more than one side of the structure is combined to generate a 3-D image of the structure. For example, in some embodiments, a volume of the structure has two surfaces (e.g. at least two surfaces) where data is collected from the first surface and the second surfaces where tissue of the structure is between the two surfaces. In some embodiments, measurements and/or image/s are generated from the data collected from the first and second surfaces e.g. as measurements of a thickness of the structure between the first and second surfaces.

Combination of data collected from different sides of a structure provides potential advantages for image accuracy and/or precision. Certain well-known arrangements of electrodes place some of them inside the lumen (e.g., on a catheter inside the lumen), and some of them outside (e.g., attached to the skin of the patient). In these cases, the single internal set of electrodes is used to measure the electrical field, since it is the only set placed to accurately discriminate influences due to the features of interest for imaging (that is, to discriminate effects on the electrical field affected by the imaged structure). Electrodes inside the lumen may have line of sight to the lumenal wall and structures arranged on it. Electrodes outside the lumen are generally placed so that electrical fields have to pass through one or more additional tissue structures before reaching the lumenal wall.

To form an image in the general case, electrical field measurements are transformed according to a transformation which is itself calculated based largely on values of the measurements themselves, together with a set of constraining assumptions. While these inputs greatly restrict the reasonable possibilities for the transform (and thus, the resulting image), there are some general sources of uncertainty and/or error. For example, the measurements themselves may be noisy, the constraints used may be based on assumptions which are not strictly (but only approximately) correct, and/or some of the constraints may be contradicting, and the image represents some compromise between them.

With respect to reducing such uncertainties and/or errors, there are both general and specific potential advantages to obtaining electrical field measurements from intra-lumenal positions on at least two sides of a tissue structure.

Having more measurements, even from one side alone, potentially reduces sampling noise by averaging. However, additional measurements introduced from a single side are likely to share certain systematic errors in common, preventing them from being reduced by averaging. A potential advantage of measuring from two sides of a tissue structure is that systematic errors particular to a first side (e.g., due to uncompensated environmental influences) will not exist on the other side. Even something as simple as averaging separate imaging results of the two sides together potentially reduces these unshared systematic errors. In the case of location clouds, averaging optionally comprises determining locations associated with corresponding features determined from each of the two sides. Corresponding features are optionally found to correspond by having same relative positions, e.g., positions relative to a surface that the location cloud determined from each side defines, though not necessarily same absolute positions in space. Additionally or alternatively, artifacts due to certain kinds of systematic error are potentially distinguishable when there are two different image results to compare. For example, if each of two images show a certain high spatial frequency feature (a “bump”, for example), then it is optionally accepted as a valid feature. If only one of them shows the feature, then, optionally, it is considered to be artifactual, and can be indicated as such, and potentially removed entirely from a combined imaging result.

In some embodiments, the information that different readings were obtained from electrodes that resided on two different sides of the imaged structure is used to place the transformation of measurements to locations under a constraint—that both measurement sets are measurements of the same structure. Averaging (e.g., of intermediate image results, for example, in the form of location clouds and/or images obtained from each data set separately) applies this constraint as if it is assumed that errors are assigned equally to both sides (albeit weighting could be used to emphasize one image more than the other). Feature removal applies this constraint as if it is assumed that features seen only in one of the images are artifactual. Another type of constraint can be applied when the locations of measurement on the two sides of the structure are at a known distance from each other (for example, located on a same catheter probe). This potentially assists, e.g., in setting scaling and/or distance factors for the images. For example, a large but distant feature and a small but nearby feature may be potentially confused with each other as two equally plausible solutions to the inverse problem, consistent with data available. By imaging the same feature from two sides at a known total distance between the imaging electrodes, two images may become available, each of the same target having its size. Insofar as that size is constant in the two images—and the possible target distance from each measurement sight constrained by their known distance from each other—the size is also constrained to have a particular value. In some embodiments, this constraint may be applied without having two images, but as a cost function, for example, that “punishes” solutions where the two sides of the same structure have different sizes.

More generally: since the two images are each of the same structure, they may be constrained to be consistent with each other, even though they are otherwise subject to some independent influences. This potentially excludes the number of “consistent but incorrect” solutions to the inverse problem which convert measurements into images. Similarly, since the two sets of electrodes provide information on two sides of the same structure, a constraint may be set to require the readings of the two sets of electrodes be consistent with each other, for example, to be transformed to structures of the same size.

In some embodiments, measurements are collected during a valve treatment procedure. For example, during placement of a valve clip (e.g., attachment of a mitral valve clip to the mitral valve), or a device for occluding the left atrial appendage. Where, in some embodiments, imaging generated from electrode measurements is used to guide positioning of the valve clip. In some embodiments, the valve clip and/or a delivery device for the valve clip (e.g. catheter to which the valve is coupled) includes one or more electrodes. In another example, during placement of a plug, clip, or other device to close a left atrial appendage (LAA), the structure being imaged is optionally the ostium of the LAA—with one set of electrodes inside, and one outside. In some embodiments, the region is mapped, providing a detailed map of the ostium. Optionally, the catheter within the LAA is removed, the other remains outside, guiding the closure with reference to the detailed map generated using the two sets of electrodes.

In some embodiments, measurement data includes data measured using two or more different measurement modalities. For example, in some embodiments, measurements include electrical measurements and ultrasound measurements. In some embodiments, a first side is measured using electrical measurements and a second using a second modality e.g. ultrasound.

In some embodiments, measurements are collected using fields emitted by electrode(s) which are not located in a vicinity of the tissue to be measured and/or are not in contact with the tissue. For example, in some embodiments, measurements include measurement of deformation of field/s within the tissue to be measured. For example, where measurements include one or more feature as illustrated and/or described in U.S. Provisional Patent Application No. 62/546,775 which is hereby incorporated by reference in its entirety.

Exemplary Local Spatial Position Constraints on Reconstruction

In some embodiments, reconstruction (and/or in particular transformation generation) of a body cavity shape and/or navigation in a body cavity may be obtained by first assuming local spatial position constraints which are consistent with the physical conditions applying to individual sets of measurements (like the known relative distance of measuring sensors at the time the measurements were taken). In some embodiments, this assumption is combined with use of a multidimensional scaling (MDS) algorithm. MDS algorithms refer to a class of algorithms wherein objects (in some embodiments, measurements of voltage) are placed in an N-dimensional space (e.g., as described herein, the three dimensional space of a body cavity) so that between-object distances are preserved as well as possible (given all other, potentially competing, constraints). In some embodiments, the geometrical configuration of sensors on an intrabody probe provides the between-object distances, allowing an MDS approach to be used for reconstruction of a body part. In some embodiments, the configuration is fixed (e.g., a rigid catheter section). In other embodiments, the configuration may be flexible (e.g., a flexible probe section or multiple probes), however, there may still be useful constraints on the relative positions of probe sections, such as possible distances between sensors due to probe flexibility and deformability limitations and/or other properties. In addition, estimations of geometrical properties of the probe (or probes) and interrelations between sensors carried thereon may be used, for example, probe position values and/or sensor position values provided by position sensors and/or restrictions on movement provided by nearby structure and/or based on possible speed of movement of parts of the probe. It is noted that many of these constraints are local (e.g., relate to volumes with a diameter of less than 50%, 20%, 10% or intermediate percentages of a largest dimension of the reconstructed shape). In some embodiments, more global constraints are used, for example, on an overall shape of the transformation, on a uniformity of the transformation (e.g., as compared to a generic transformation based on generally expected behavior of electric fields in the body) and/or based on expected distances between closest simultaneous measurements.

In some embodiments of the invention, several sets of measurements x are obtained in X; each set x being made up of a plurality of measurements xᵢ, xⱼ, . . . measured simultaneously by different sensors i, j on a same probe; and with distances (e.g., or other geometrical constraints) between at least some of the sensors being known or estimated (e.g., including bounded), so that the distances can be used as a constraint. Moreover, in some embodiments, more than one measurement is made from each sensor (e.g., measurements of different electrical fields, e.g., of fields having different frequencies), so that the set of measurements in total includes, e.g., xᵢ_{_{1,2, . . .}} ,xⱼ_{_{1,2, . . .}} , . . . . It is noted that these constraints may be recalculated as part of the reconstruction. Measurements in a set are optionally taken substantially simultaneously, i.e., while the probe remains in substantially the same position. Moreover, in some embodiments, the different measurement locations on the probe optionally have known spatial relationships to one another, which comprise, in some embodiments, local spatial position constraints. Reconstruction of the body cavity shape may be guided based on these known spatial relationships; for example, in some embodiments, a transform function T(x) on a each member of group of measurements X comprising the set of measurements x may be calculated such that |T(Xᵢ)−T(Xⱼ)|≈dᵢⱼ; dᵢⱼ; being the distance between electrode, and electrodeⱼ.

For example, in some embodiments, the electrodes are each at a known distance and/or angle from one another due to a fixed geometry of the intrabody probe to which they are mounted. Alternatively, in some embodiments, electrodes are in variable relative positions, and the variation accounted for based on information such as parameters of deployment (e.g., how expanded a basket-shaped intrabody probe is at a moment of measurement), and/or on further measurements (for example, of force as an indication of probe deformation, inter-electrode conductance as an indication of inter-electrode distance, etc.). Optionally, additional constraints on the relative orientation of the measurement locations are also used. Such constraints are optionally known, for example, from geometrical/anatomical constraints on the procedure itself.

Optionally, measurements in each set are substantially simultaneous. Herein, “substantially simultaneous” should be understood to mean that the measurements of each set may be obtained:
actually simultaneously (i.e., with partially or wholly overlapping measurement periods),
close enough in time that motions of the intrabody probe during acquisition of the set can be neglected, and/or
close enough in time that skew due to small movements during sampling of a set of measurements can be dependably factored out and/or adjusted for if necessary (e.g., by use of time-weighted averaging of time-adjacent samples).

Optionally, a collection of measurements is considered as a set of measurements mutually constrained in relative position (e.g., fixed at particular relative distances and/or relative angles, at variable but known distances or angles, for example by use of an encoder, etc.), without a requirement for substantial simultaneity of measurement. For example, multiple measurements at multiple times from an intrabody probe are optionally taken while a portion of the intrabody probe remains anchored at one or more regions. Relative movements of other intrabody probe portions, assuming they are known (by use of a movement encoder, for example) can then be applied to determine a relative position constraint. These measurements are optionally related to one another through use of the fixed anchor and the known bending parameters to provide calibration. It can be understood from this, and it should be understood to apply generally, that a measurement (also known as a “measurement sample”) optionally is treated as a member of a plurality of “sets” of measurements, where members of each set may be related to one another through application of different mutual position constraints.

For simplicity, and for purposes of description herein, sets of simultaneous measurements from corresponding electrodes of a fixed-shape probe are often used in examples. However, it should be understood that other configurations of sensors, and/or other methods of obtaining a spatially calibrated “ruler” to constrain distances between them are optionally used in some embodiments of the present invention. In some embodiments, the constrained distances may be used to ensure that the target shape is reconstructed so that the distance between the electrodes (e.g., in mm) is kept approximately the same all around the reconstructed shape, even if the difference between their readings (e.g., in mV) changes substantially from one place to another. For example, in some embodiments, the length of the catheter is reconstructed to be the same within ±15% even though the voltage gradient between the same electrodes changes by a factor of 10 or more.

Herein, voltages measured substantially simultaneously by two electrodes separated from each other by a fixed distance (e.g., because they are fixed to a rigid probe portion), may be referred to as sister measurements; the locations assigned to such measurements may be referred to as sister locations; and the distances between sister locations may be referred to as sister distances. The fixed distance itself may be referred to as a desired sister distance.

In some embodiments, a transform function to be found is defined as comprising two terms: one which gives a roughly scaled transformation of V-cloud measurements into an R-cloud, and a second which applies displacements to that roughly scaled R-cloud. The second term potentially helps overcome at least some of the electrical field non-linearities and/or non-orthogonality which may exist in the roughly-scaled transformation.

The rough-scaling term of the displacement approach of some embodiments of the invention can be understood, for example, by envisaging each measurement set x of the measurements X to be first “copied” from a coordinate system in a measurement space, wherein each of the measurement space axes is, e.g., an axis of measurement values for one of a respective plurality of crossing electrical fields; to a coordinate system in a physical space, wherein different positions along the axes represent different locations in physical space. This copying may be carried out with a different scale along each axis; for example: a voltage difference of 1 mV measured along a horizontal axis in the measurement space may correspond to a distance of 3 mm along the horizontal axis of the physical space, and a voltage of 1 mV measured along a vertical axis in the measurement space, may correspond to a distance of 2 mm in the physical space. In notation form, the voltage points X may be envisaged to be first “copied” to initial location points Y, e.g., by a scaling transformation Y=diag(a)X, where a is, in some embodiments, a vector comprising scaling coefficients a=(aₓ,a_{y},a_{z}), with units of distance/measurement (e.g., mm/mV). diag(a) indicates the matrix diagonalizing vector a. With the addition of a displacement term W, the initial location points diag(a)X are displaced by displacement W to have the proper local scaling (i.e., to make sister distances in Y optimally correspond to the known distances between the sensors). It is noted that while the axes in the physical space may be orthogonal, this does not limit the method to embodiments where the fields themselves are orthogonal to each other, or even close to orthogonality (e.g., the axes may be, for example, 20 degrees or more off axis, for example).

The axes in the physical space are provided as a convenient means for describing locations in space, and the transformation from the measurements to the positions by the rough-scaling term is arbitrary. Still, the more orthogonal are the fields, the less arbitrary is this transformation, and the computational effort required to find the optimal transformation may be smaller. In some embodiments of the invention, the rough-scaling term is mainly used for transforming the data from units of voltage (or other measurement) to units of length. In addition, if the data implies a need to stretch the reconstruction along some direction, the rough-scaling term can allow doing so using a smaller number of actions than would be required if only W was available for applying such stretching (e.g., in case the rough-scaling term was predetermined to be the same for all the fields.

The displacement term W can be decomposed in different ways in order to guide the search for the individual displacements that make it up. In some embodiments, accordingly, the displacement W is expressed as a multiplication of two matrices: W=UW′, with U being a representation of X in a coordinate system “natural” to X, and W′ being a matrix of coefficients (displacement coefficients) which give the magnitude of displacements applied within the same “natural” coordinate system, also referred to herein as a coordinate system that preserves the “intrinsic geometry” of X.

This intrinsic geometry, in some embodiments, is defined as comprising a set of linearly independent features (referred to as characteristic vectors or eigenvectors v of a similarity matrix, reflecting similarity between sampled measurements) which “sum up” (after individual scaling of the eigenvectors v, each by its eigenvalue) to produce an equivalent representation of X.

Decomposition of X into eigenvectors, in some embodiments, has the effect of separating features according to their spatial frequencies. This property is optionally used in relation to maintaining spatial coherence, for example as discussed hereinbelow.

In some embodiments, a kernel K is defined as a matrix that expresses a measure of the distances between each pair of measurements:

$${K}_{i , j} = K ( {x}_{i} , {x}_{j} ) = {e}^{\frac{- {‖ {x}_{i} - {x}_{j} ‖}^{2}}{2 {σ}^{2}}}$$

This form of a kernel is optionally referred to as a radial basis function kernel, and is an example of a similarity matrix. The sigma parameter is a free variable, which optionally is set to be about 0.1. Optionally, the kernel K is normalized to a normalized kernel \tilde{K}, for example by one of:

$$S = diag {\sum }_{j} {K}_{i , j}$$

$$\tilde{K} = \frac{K}{S}$$

$$or$$

$${S}_{i} = diag {\sum }_{j} {K}_{i , j}$$

$${S}_{j} = diag {\sum }_{i} {K}_{i , j}$$

$$\tilde{K} = \frac{K}{\sqrt{{S}_{i} {S}_{j}}}$$

$$or$$

$$S = diag ( K \cdot n )$$

$$\tilde{K} = {S}^{1 / 2} K {S}^{- 1 / 2}$$

$$wherein n = [ \begin{matrix}1 \\ 1 \\ … \\ 1\end{matrix} ]$$

The normalized kernel \tilde{K} is decomposed to find U, for example, using the Graph Laplacian, such that for the k most significant eigenvectors u:

The eigenvector matrix U is: U=[u₁, . . . uₖ]
The eigenvalue matrix V is: V=diag([λ₁, . . . λₖ])
And the decomposition satisfies: \tilde{K}u=λu

Putting the terms just described together results in an X (measurement) to Y (position) transformation which may be expressed by the equation Y=diag(a)X+UW′.

Each set of a and W′ provides a configuration that gives a generally different transformation of X to Y. To find the transformation that provides a best fit between sister distances and the desired sister distances (e.g., known distances between the sensors on the probe), a penalty may be associated with each deviation of the sister distances from the known distances, and this penalty minimized by known minimization procedures. Other penalties described herein are also optionally applied, e.g., by addition to the penalty on the difference between sister locations and known distances between sensors on the probe. A choice of a and W′ with a minimal penalty result gives, from the point of view of the algorithm and its particular cost function, the “correct” Y from the given X.

Coherence Constraints on Reconstruction

In some embodiments, reconstruction of a body cavity shape and/or navigation in a body cavity using such a reconstruction may be obtained by imposing coherence constraints, e.g., a coherence model, on a transformation, a set of measurements and/or a set of geometrical positions after transformation.

In some embodiments of the invention, the coherence constraints are added to constraints on relative positions assigned to sensors (e.g., to the above-mentioned constraints of having sister distances similar to desired sister distances). An example for a coherence constraint may be that two measurements made at nearby regions in space are assumed to produce measurement values which are also “nearby” in the measurement space under some metric (e.g., change in voltage of a certain number, for example, 5, 3, 2 of the crossing fields is less than, for example, 30%, 20%, 10% or intermediate percentages). Similarly, the transformation of measurements to locations may be constrained so that every two measurements of “nearby” values are transformed to locations close to each other, under some metric. In some embodiments of the invention, “nearby” is defined as a function of the range of the reconstructed volume, for example, a distance of less than 30%, 20%, 10%, 5% or intermediate percentages of a maximum dimension of the reconstructed volume. Optionally or additionally, “nearby” is defined as a function of time (e.g., how long would it take or did it take a probe to move between positions, for example, 30 ms, 20 ms, 10 ms, 1 ms or smaller or intermediate times. Optionally or additionally, nearby is defined as function of the probe geometry, for example, less than 10×, 5×, 2× or intermediate multiples of a smallest or largest distance between electrodes on a catheter.

It is noted that a same constraint (e.g., coherence or known distance deviation) may be considered as a single constraint (e.g., applies to all the data) or as a plurality of separate constraints (e.g., applies separately to each data point or pairs thereof. In some embodiments, processing is simplified by aggregating constraints so that they are treated as one for optimization purposes. For example, a distance constraint may be defined as a single constraint on all distances and electrode pairs, which may be relaxed or enforced as a single constraint.

A coherence criterion may be set to require that the transformation transforming measurements to locations would be smooth, that is, that small differences in measurements in one place in the measurement space will not result in much larger difference in locations than in a neighboring place. Since sensors on the probe are at neighboring places, such a constraint may be applied on sister distances, that is, that sister distances don't change abruptly from one place of the probe to another. This may be achieved, for example, by using a cost function penalizing transformation making use of high frequency components, and the overall penalty (also referred to herein as “cost”) may be minimized (by reducing the contribution of high frequency components to the transformation) in order to find a coherent transformation. It is noted that even if a transformation is smooth, it may vary, for example, by a factor of 2, 3, 4 or more in one or more dimensions, at measurement locations that are not adjacent (e.g., >10% of volume diameter away).

For example, a coherence criterion may be set by setting a penalty to each of the k eigenvector components of matrix U, and this penalty may be higher as the frequency of the component is higher, and increase as the displacements along this component are larger. This way, distributions that result from transformations that include only displacements along low frequency components would nearly not be penalized, and those that result from transformations that include displacements along components of very high frequencies will be penalized heavily. A minimization procedure may be applied to minimize the penalty, to find a transformation that results in sister distances that change smoothly (e.g., transformations with displacements mainly along small frequency components), which is an example of a coherence criterion. Additionally, a coherence criterion is optionally influenced by the direction of the voltage gradient (i.e., a smaller change in gradient direction is “more coherent”), and/or by the rate of change in the gradient itself (and/or its direction) and/or any higher order gradient derivative.

Additionally or alternatively, in some embodiments, coherence of a transformation result is enhanced by how many eigenvectors are used (the value of k). In some embodiments, k is around 50-250; optionally or alternatively, k is a value around 20-25% of the total number of measurement vectors x in X. For example, if only the k lowest-frequency components are used, the larger is k, the less coherent, potentially, is the transformation. However, larger k (that is, allowing transformations along more components of U) results in larger flexibility and better chance to minimize other terms in the cost function (e.g., the requirement for sister distances that are similar to the desired sister distances).

A metric by which distances are measured for defining coherence and/or sister distances, can be, for example, the Euclidean distance. In some embodiments, the metric may be a “natural” distance, for example, an Euclidian distance defined in the natural geometry of the measurements cloud, that is, over the components of the U matrix. In some embodiments, the metric may be a distance in a measurement-defined vector space (i.e., a vector space comprising a plurality of different measured parameters as vector components), but may also be more involved than that.

Optionally, the coherence constraint can be expressed as ΔXᵢⱼ∝ΔYᵢⱼ where ΔXᵢⱼ is a change between two locations i,j of measured values in X (for example, changed measurements of voltage with respect to a plurality of crossed voltage gradient-defined axes), and ΔYᵢⱼ is a change in the spatial position (e.g., distance, under a suitable metric) between the two locations i,j, within the body cavity to be reconstructed, Y.

The proportionality sign ∝ should be understood to refer to any suitable coherence metric and/or algorithm (coherence model), not necessarily constant uniform proportionality. For example, a proportionality parameter is optionally allowed to vary (e.g., with a factor of at least 2, 3, 4 or intermediate or greater values) over the domain of measurement values. In some embodiments, the coherence model allows the proportionality parameter to vary smoothly, and/or according to a model of expected behavior, e.g., varying smoothly everywhere except near the edges or other particular zones of the mapped space.

As mentioned, in either physical space or measurement space, distances are not necessarily direct Euclidean distances. In some embodiments, for example, the measurements may form a measurement cloud (in some measurement vector space, for example), and the spatial positions to which the measurements are transformed may form a position cloud (also referred to herein as a “location cloud”). In some embodiments, a natural distance between two measurements may be defined as the length of the shortest path that goes between the two measurements only through the measurement cloud. A path going only through a cloud is referred to herein as an intra-cloud path. Similarly, a natural distance between two spatial positions may be defined as the length of the shortest path that goes between the two spatial positions only through the position cloud (that is, the shortest intra-cloud path in space). In some embodiments, the measurement cloud may be segmented, in the sense that it includes distinct segments; for example, a central segment connected to each of a plurality of peripheral segments.

The peripheral segments may be interconnected only by pathways passing into the central segment from one segment, and back out of it to the other. In such embodiments, two peripheral segments may have points (e.g., measurements) that are nearby in the Euclidean sense, but the natural distance between them is long, as every intra-cloud path between them goes via the central segment. In such embodiments, measuring coherence using natural distances may preserve the segmentation of the measurement cloud, so that the position cloud remains similarly segmented. That is, a transform requiring coherence in terms of natural distances may transform a segmented measurement cloud into similarly segmented spatial positions cloud. Such a transform (whether based on intra-cloud coherence or preserving the segmentation by different means) may be referred to herein as a segmentation preserving transform. A segmentation preserving transform is potentially suitable to preserving features of heart chambers; for example, for preserving the pulmonary veins connected to the left atrium and separated from each other.

An example of a segmentation preserving method of transforming a segmented measurement cloud into a similarly segmented position cloud may include assigning each measurement to a segment in the measurement cloud; and transforming each measurement to a position in a segmented spatial position cloud requiring that measurements assigned to a same segment in the measurement cloud are transformed to a same segment in spatial position cloud and measurements assigned to different segments in the measurement cloud are transformed to different segments in the spatial position cloud. Such segmentation preserving method may replace a Euclidean-distance based coherence condition, or be used in addition. For example, in some embodiments, the coherence may be primarily based on Euclidean distances, with segment preservation used to protect against segments coalescing, e.g., by disallowing influence on the coherence model by differences between points whose Euclidean distance is sufficiently shorter than their natural distance.

Combination of Local Scaling and Other Constraints

In some embodiments, the approaches of local spatial constraint (e.g., on sister distances) and a coherence-related constraint are used in a combined method of transformation (e.g., generating a transformation meeting these constraints). Outputs of each are optionally reconciled by use of an error (equivalently referred to as cost, penalty or “energy”) reducing weighting scheme, for example as now described.

Initially, in some embodiments, the detailed, or optionally even the overall geometry defined by a “true” body cavity shape Y is unknown, but still, a useful approximation may be obtained by a transformation that transforms the measurements according to the applied constraints. The target for “usefulness” of the approximation is optionally dependent on the particulars of the procedure, and even of particular tasks within the procedure; and there can be a plurality of criteria for evaluating the accuracy of reconstruction, optionally applied simultaneously (e.g., as concurrent penalty weightings), and/or separately (e.g., to obtain reconstructions optimized for different sets of penalty weights). In some embodiments, for example, the target for “useful approximation” is to be able to place adjacent small lesions next to each other within some relative margin of error as part of an ablation procedure; for example, an error within 0.5 mm, 1 mm, 2 mm, 4 mm, 8 mm, or some other intermediate margin of error. Additionally or alternatively, another target for useful approximation is positioning a linked chain (or other grouping) of small lesions within some margin of error relative to landmarks of a target tissue; for example, an error within 1 mm, 2 mm, 4 mm, 8 mm, or another intermediate margin of error.

In some embodiments, the measurements are known to be obtained by sensors fixed at known distances from one another, e.g., because they were obtained from a plurality of different sensors positioned at fixed distances on an intrabody probe. However, the known relative position constraint is not limited to the use of sensors arranged in a linear, ruler-like configuration. For example, in some embodiments, the sensors are arranged in pairs, where each two electrodes in a pair are so close that the catheter cannot practically fold between them, but the inter-pair distances are large enough so that the catheter may fold between pairs. In such an embodiment, intra-pair distances may be known, and inter-pair distances may be unknown. It has been found that the intra-pair distances may be sufficient for obtaining useful approximations. The cost function optionally comprises another constraint based on distance and/or relative angle of measurements. Expressed in notation, for example, the measurements are position-constrained such that a transform yielding distance |T(Xᵢ)−T(Xⱼ)|=ΔY′ᵢⱼ can be found, with a result that is potentially a good approximation of the actual distance. Optionally, the transform is found by a process of “energy” or error/penalty reduction as just outlined.

Considering local spatial calibration (e.g., MDS-used, and/or sister distances-based) constraints alone, the relative positions of each separate set of measurements (e.g., a set of measurements taken at different times and/or at different locations in the target) are unlinked. The measurements themselves are subject to measurement noise. Therefore, there may remain uncertainty about how different measurement sets should be related to one another in space.

In some embodiments, this problem is alleviated at least in part by incorporating into a reconstruction algorithm assumptions about coherence between distances in the measurement space and distances in the physical space. Optionally, coherence and local spatial calibration constraints are weighted relative to each other to achieve reduced transformation error and/or reconstruction (in general) error.

Conceptually, the weighting can be thought of as allowing mutual position constraints to act as a ruler, measuring differences between positions in units of distances between electrodes, and influencing and/or partially overriding the local conditions of coherence. Conversely, the constraint of coherence may help to assign different sets of measurements to positions in space, while mitigating distorting effects of measurement noise. As more measurements are made, the limits of the body cavity in which the probe is moving will limit the extent of movements, so that the reconstruction Y′ potentially grows to more closely resemble the actual shape of the cavity Y (herein, the notation Y′ may be used to designate a reconstruction in contexts where its distinctiveness from the actual cavity shape is being emphasized).

In some embodiments, for example, the transform T is defined as a transform that minimizes a suitably weighted joint error in satisfying both the coherence condition and local spatial constraints. For example, error with respect to local spatial constraints is optionally found from |T(Xᵢ)−T(Xⱼ)|= [symbol] Y′ᵢⱼ≈ [symbol] Yᵢⱼ, where the error is in the deviation of distances in Y′ from known real-world distances in Y (e.g., error is |Y′−Y|, or another suitable error metric). Similarly, error with respect to coherence is optionally found from [symbol] X∝ [symbol] Y″≈ [symbol] Y′, where the error is in the differences in Y′ from the coherence-modeled output Y″ (e.g., error is |Y′−Y″|, or another suitable error metric). Minimization of error is by any suitable technique, for example, statistical analysis and/or gradient descent. The symbol ≈ is used herein to show that discrepancies between the terms on its both sides (in this case, between T(x) and Y), are minimized by use of a suitable reconstruction procedure, although equality cannot be guaranteed.

In some embodiments, a reconstruction of Y is produced exclusively or almost exclusively based on sensor measurements, their known distances, and optionally an assumed coherence model.

In some embodiments, a reconstruction of Y is produced exclusively or almost exclusively based on imposing local spatial position constraints and optionally coherence constraints on a set of measurements.

In some embodiments, a coherent transformation may be obtained by a method using spectrum decomposition, for example, by a diffusion map algorithm. In some embodiments, such a transformation may be segmentation preserving. For example, embodiments are described herein using the concept of displacement optionally are modified to preserve coherence by the selection and/or weighting of components, along which the displacements occur, according to their spatial spectrum frequency.

Each constraint may be embodied by applying a penalty to a transform insofar as the transform violates the constraint. For example, the constraint to have the sister distances as accurate to their known distance as possible may be embodied in a “penalty” applied to transformations that generate sister distances that deviate from the known “ruler” length: the larger the deviation—the larger the penalty. Thus, adjusting the transform to reduce the penalty applies a criterion for reducing the variability in the sister distances. In some embodiments, reducing variability in sister distances reduces differences between sister distances and the desired sister distances. In some embodiments, a cost function penalty that encourages having the sister distance as similar as possible to the known distance will be in addition to a cost function penalty that encourages the sister distance to be kept as constant as possible across the transformation. In some embodiments, a cost function penalty that encourages minimizing differences between sister distances and the desired sister distances may result in reduced variability of the sister distances without posing an explicit constraint on the variability.

A coherence constraint may be, for example, that W is smooth; for example smooth in the sense that if it is represented as a combination of displacements along linearly independent spatial components of different spatial frequencies, it includes only or primarily displacements along components of low spatial frequencies.

Eigenvectors of high frequency are typically more influenced by noise in the measurement cloud, than by major structural characteristics of the cloud. Thus, taking into account only the eigenvectors associated with the lowest frequencies allows grasping the major structure of the cloud while cleaning part of the noise, and ensures, for example, that the displacement UW′ would be of at least some smoothness.

Furthermore, reducing the contribution of eigenvectors of the highest frequencies reduces the dimensionality of the problem, as the potential displacement W′ is limited to displacements along the low frequency eigenvectors (and linear combinations thereof). This may be thought of as defining in the cloud some sub-clouds (which may also be referred to as segments) that together reproduce the major structural characteristics of the cloud, and limit the displacements to be within these sub-clouds. Therefore, this method may be considered segmentation preserving.

The constraint to have the displacement change smoothly, and in a coherent manner, may be achieved by applying a “penalty” to the various components of the displacement: the higher the spatial frequency of a component, the larger is the penalty to its contribution. Once a displacement W that minimizes the overall penalty (e.g., a sum, optionally a weighted sum, of the penalty for sister distance variability and the penalty for high spatial frequencies) is obtained, it may be used to displace the initial locations to their new locations, which represent a location cloud that may be used for reconstruction of the body-part. Going from a location cloud (e.g., a set of geometric positions) to a reconstruction (i.e., a model where the points in the location cloud are interconnected to form a mesh that defines outer borders to the cloud) are known in the art and are not generally a subject of the present disclosure. An example method may be found, for example, at Bernardini, Fausto, Joshua Mittleman, Holly E. Rushmeier, Cláudio T. Silva and Gabriel Taubin. “The ball-pivoting algorithm for surface reconstruction.” IEEE Transactions on Visualization and Computer Graphics 5 (1999): 349-359, the disclosure of which is incorporated herein by reference. Therefore, the terms location cloud (or R cloud) and reconstruction are used herein interchangeably. Finding W that minimizes the penalty may be carried out using standard minimization procedures.

In some embodiments, the coherence criterion is implied using the intrinsic geometry of the V-cloud, and need not be specified as a separate mechanism in the operation of the algorithm. This may be achieved, for example, by defining the smoothness criterion (which gets a larger penalty the larger it is) as W^{T}VW, where V is a diagonal matrix of the eigenvalues that correspond to the eigenvectors making up U.

Optionally, a few further conditions are set to guide the reconstruction process—for example, broad assumptions about the orientation and voltage ranges of electromagnetic fields being measured, positions of landmarks, and/or global constraints on positions and/or orientations which the intrabody probe can physically reach based on its size, flexibility, entry point to a chamber, etc. In some embodiments detailed initial conditions are set for the reconstruction. In some embodiments of the invention, such initial conditions do not include a reference point or frame which is used to define the positions of measurements relative to the point, before transformation and/or not used as part of the transformation.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Example of a System

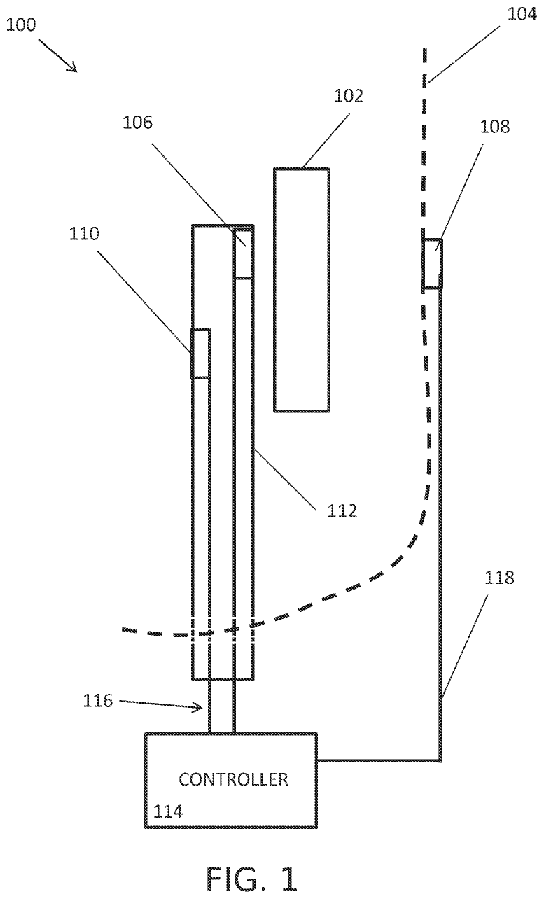

FIG. 1 is a simplified schematic of a system 100 for imaging tissue 102 within a patient 104, according to some embodiments of the invention.

In some embodiments, the system includes at least two electrodes 106, 108. In some embodiments, a first electrode is positioned within patient 104, for example, in proximity to tissue 102 to be imaged.

In some embodiments, a second electrode 108 is positioned in contact with an outer surface of the patient, for example, in contact (e.g. electrical contact) with a patient's outer skin surface.

Alternatively, in some embodiments, the system lacks an electrode in contact with an outer surface of the patient, and a second electrode 110 is positioned within the patient. Where, in some embodiments, the first electrode 106 and second electrode 110 are part of an imaging tool 112. In some embodiments, the system includes 2-50,2-20,2-10, or 2-8, or 2-5, or lower or higher or intermediate ranges or numbers of electrodes.

Alternatively, in some embodiments, the system includes two or more electrodes 110, positioned within patient 104, and one or more electrode 108 outside the patient (e.g. in contact with an outer skin surface of the patient). In some embodiments, electrode(s) 108 include one or more electrode (e.g. pad electrode) in contact (e.g. electrical contact) with a patient's skin.

In some embodiments, electromagnetic field/s are generated in tissue to be measured e.g. within the heart. In an exemplary embodiment, at least three electromagnetic fields are generated, where the fields have crossing orientations (i.e. are not parallel to each other). In some embodiments, the electromagnetic fields differ in frequency. In some embodiments, the fields are generated using one or more pad electrode attached to the patient's skin.

In some embodiments, the fields are measured by each of at least two electrodes that reside on the catheter at a known distance from each other. In some embodiments, voltage is measured. In some embodiments, other variable is measured, e.g. impedance. In some embodiments, a V→R transformation is used to reconstruct a position of the electrodes from the measurements.

Where, in some embodiments, field generation and/or measurement/s and/or reconstruction use one or more feature as described and/or illustrated in International Patent Application No. IB2018/050192 which is herein incorporated by reference in its entirety.

In some embodiments, one or more electrode positioned within patient e.g. electrode 106 and/or electrode 110 are ring electrodes. Where, in some embodiments, imaging tool 112 includes catheter onto which the electrodes (e.g. ring electrodes) are mounted and/or attached.

In some embodiments, system 100 includes a controller 114. In some embodiments, controller 114 has a data and/or electrical supply connection 116 to electrodes 106, 110 and/or a data and/or electrical supply connection 118 to electrode 108. Where, in some embodiments, one or more connection 116, 118 includes cables and/or wireless connection.

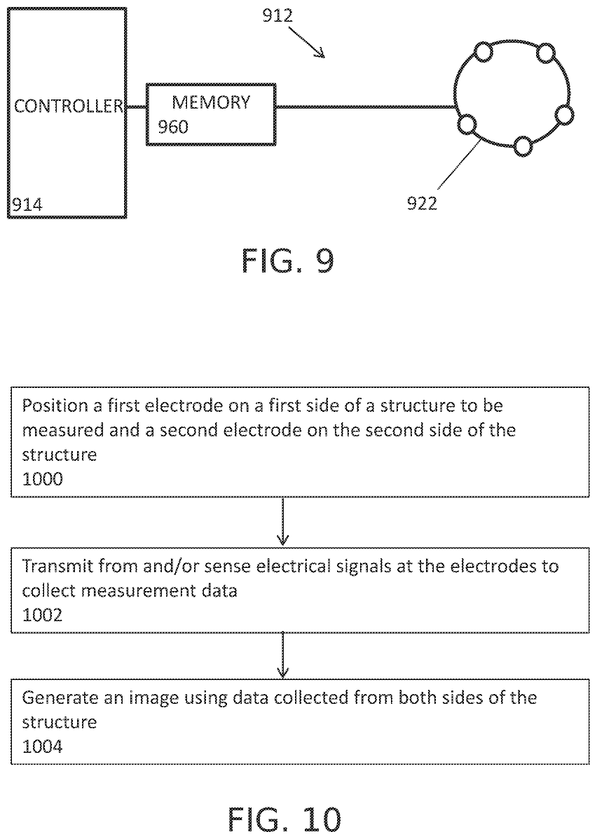

In some embodiments, controller 114 includes one or more memory and/or accesses one or more external memory e.g. as illustrated in FIG. 9 where an external memory 960 is connected to a controller 914.

### Example of a Method

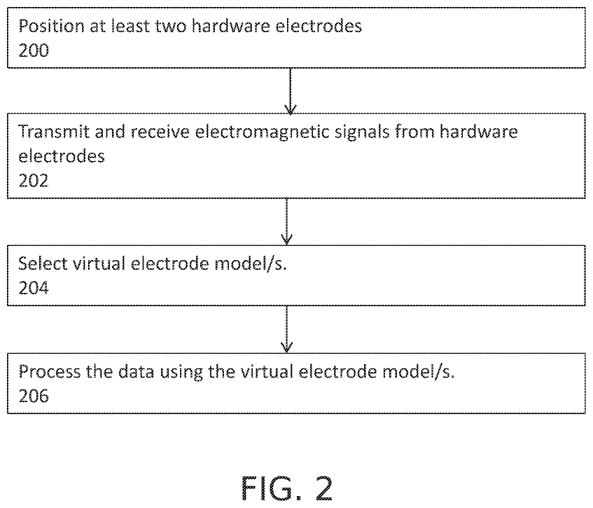

FIG. 2 is a flow chart of a method of tissue imaging, according to some embodiments of the invention.

At 200, in some embodiments, at least two electrodes are positioned.

In some embodiments, an imaging tool including at least one electrode is positioned within a patient; for example, within a chamber of the patient's heart and/or within a blood vessel. In some embodiments, the imaging tool includes more than one electrode. In some embodiments, more than one imaging tool each tool including at least one electrode are positioned.

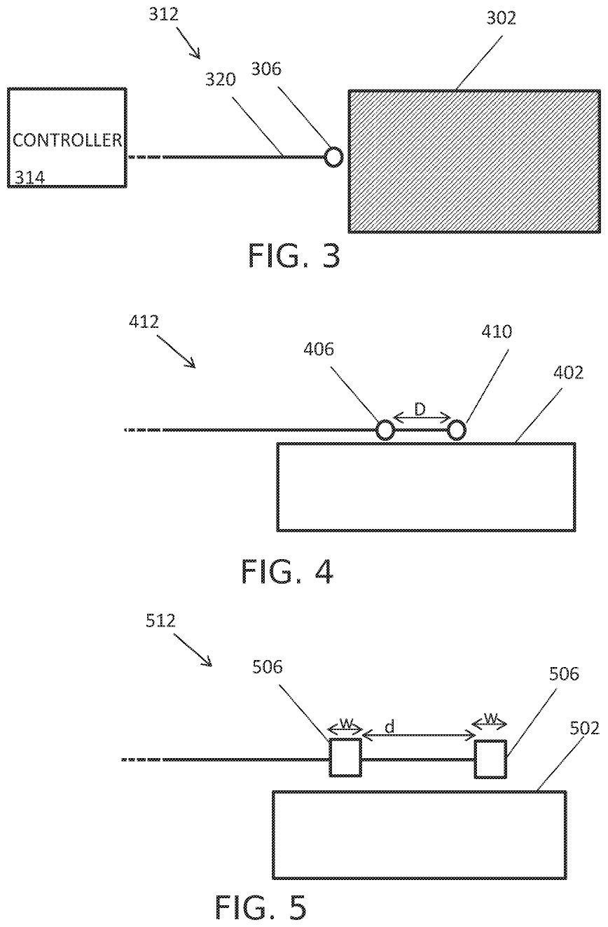

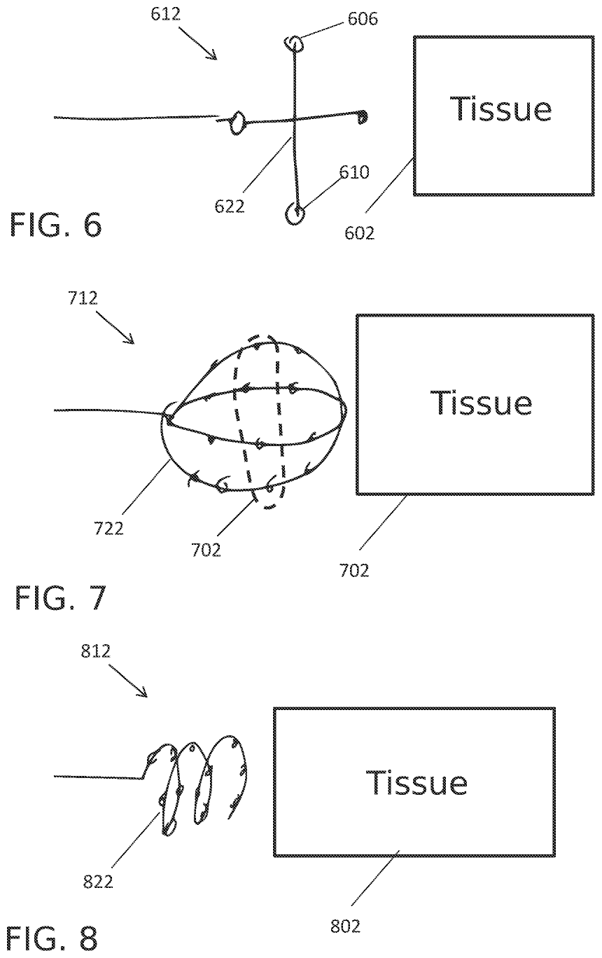

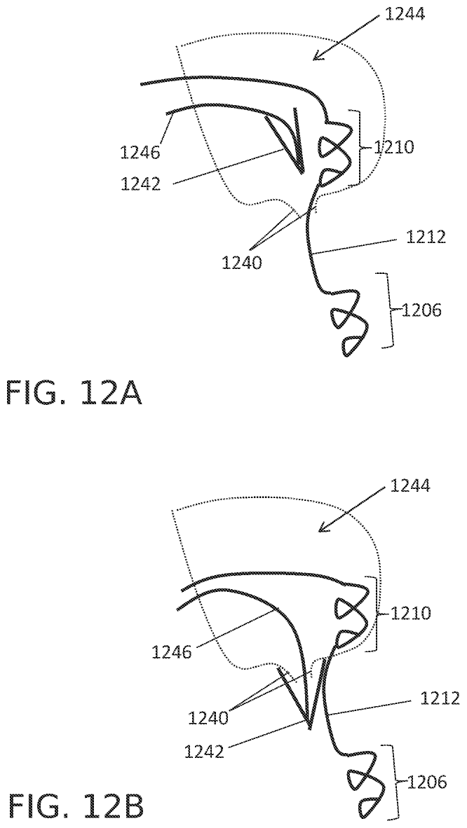

In some embodiments, the imaging tool has one or more feature as described and/or illustrated regarding imaging tool 112 FIG. 1, 312 FIG. 3, 412 FIG. 4, 512 FIG. 5, 612 FIG. 6, 712 FIG. 7, 812 FIG. 8, 912 FIG. 9, 1212 FIGS. 12A-B.

Optionally, in some embodiments, one or more electrode is positioned in contact (e.g. electrical contact) with an outer surface of the patient. For example, an outer skin surface of the patient.

At 202, in some embodiments, electrical signals are transmitted and received by the electrodes to produce measurement data. In some embodiments, two or more electrodes simultaneously transmit and/or receive electrical signals.

At 204, in some embodiments, one or more virtual electrode model is selected. In some embodiments, a virtual electrode model includes processing instructions for data from at least two hardware electrodes.

At 206, in some embodiments, the measurement data is processed using the one or more virtual electrode model (e.g. by a processor which is e.g. hosted and/or connected to a controller e.g. controller 114 FIG. 1) using the one or more virtual electrode.

In some embodiments, the processing uses a spatial relationship between one or more hardware and/or virtual electrode. In some embodiments, the processing uses a location within a volume of one or more of the hardware electrodes. In some embodiments, the spatial relationship is received from a memory which stores spatial data regarding hardware electrodes for one or more imaging tool.

In some embodiments, position and/or a spatial relationship between electrodes is measured e.g. using attenuation and/or phase shift measurements, for example, in a field generated within the tissue (e.g. as described above regarding FIG. 1).

Examples of imaging tools FIG. 3 is a simplified schematic of an imaging tool 312 including a single electrode 306, according to some embodiments of the invention.

In some embodiments, a single electrode 306 is positioned in proximity to tissue 302 to be measured. In some embodiments, electrode 306 is positioned using a catheter 320, e.g. in some embodiments electrode 306 is part of and/or mounted to and/or attached to catheter 320.

In some embodiments, imaging tool 312 is connected to a controller 314, where controller 314, in some embodiments, includes one or more feature as described regarding controller 114 FIG. 1.

FIGS. 4-5 are simplified schematics of imaging tools 412, 512 including two electrodes, according to some embodiments of the invention.

Referring now to FIG. 4, in some embodiments, tool 412 includes a first electrode 406 and a second electrode 410 where a distance D separates the electrodes.

Referring now to FIG. 5, in some embodiments, tool 512 includes a first electrode 506 and a second electrode 510 where a distance d separates the electrodes. In some embodiments, each of electrodes 506, 510 have a width, W.

FIGS. 6-9 are simplified schematics of imaging tools 612, 712, 812, 912 including a plurality of electrodes, according to some embodiments of the invention.

Referring now to FIG. 6, in some embodiments, tool 612 includes a plurality of pairs of electrodes connected by connecting elements where, in some embodiments, the connecting elements are elongated. Where, for example, electrodes 606 and 610 are connected by connecting element 622. In some embodiments, the pairs are connected by connecting their respective connecting elements. In an exemplary embodiment, tool 612 includes four electrodes each pair of electrodes connected by a connecting element, the two connecting elements connected together.

Referring now to FIG. 7, in some embodiments, tool 712 includes a plurality of electrodes (illustrated as circular dots) arranged on curved elements (e.g. curved elongated elements). In some embodiments, curved elements 722 are connected to enclose a volume which, in some embodiments, has circular and/or ovoid cross section e.g. in some embodiments the enclosed volume is spherical in shape. In some embodiments, each curved element includes more than one electrode.

Referring now to FIG. 8, in some embodiments, tool 812 includes a single curved element 822 onto which one or more electrodes are mounted and/or connected. In some embodiments, curved element is helical in shape.

Referring now to FIG. 9, in some embodiments, tool 912 includes a loop where one or more electrodes are mounted to and/or connected to the loop. In some embodiments, the loop is circular or ovoid in shape.

In some embodiments, tool 912 is part of a system including a controller 914 (e.g. which includes one or more feature as described regarding controller 114) and optionally includes a memory 960 external to controller 914.

### Examples of Virtual Electrodes

In some embodiments, a virtual electrode includes a subset of electrodes in a volume, for example from a subset of electrodes of an imaging tool. Where, in some embodiments, operation of the subset of electrodes defines features (e.g. orientation and/or size and/or shape) of the virtual electrode. Referring back now to FIG. 7, an exemplary virtual electrode 770 is constructed from four hardware electrodes of imaging tool 712. In some embodiments, hardware electrodes of virtual electrode 770 are to act as a phased array. In some embodiments, a virtual electrode acting as a phased array generates a planar beam e.g. where the hardware electrodes of virtual electrode 770 are arranged linearly with a phase delay between transmission of adjacent hardware electrodes.

In some embodiments, a virtual electrode acting as a phased array generates a more complex shaped beam, where for example, hardware electrodes of the virtual electrode occupy different planes in space. For example, in some embodiments, a virtual electrode acting as a phased array generates a beam focused on a 3-D area (e.g. around a point in space). For example, where virtual electrode phased arrays include one or more feature as described and/or illustrated in “A Novel Three-Dimensional Beamforming Antenna Array for Wireless Power Focusing” by Mohammad A. Safar and Ayman S. Al-Zayed, Mathematical Problems in Engineering Volume 2016, Article ID 7426429, which is hereby incorporated by reference in its entirety.

In some embodiments, a phased array is implemented in hardware, where a signal is sent from a first electrode and then physically delayed before being sent to a second electrode and then further delayed for additional electrode(s). In some embodiments, a phased array is implemented in software, where data is collected from each electrode and then processed.

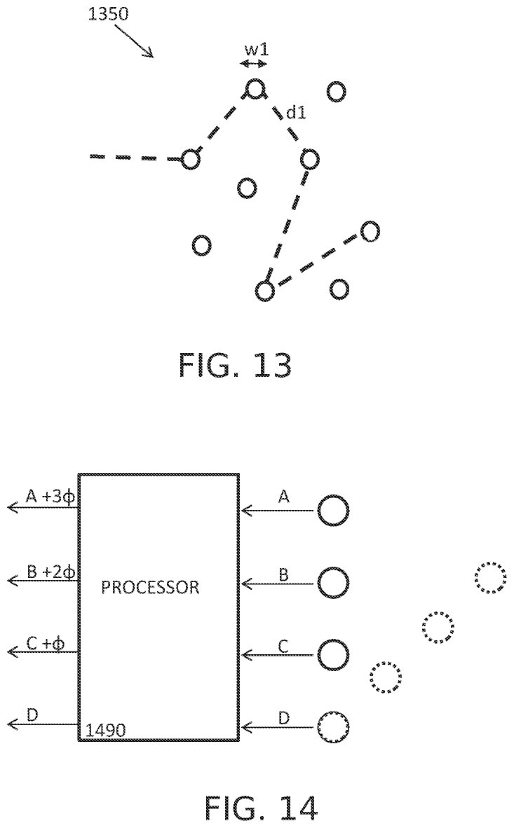

FIG. 13 is a simplified schematic of a virtual electrode 1350, according to some embodiments of the invention.

In some embodiments, in FIG. 13 hardware electrodes are illustrated as circles and a virtual electrode is illustrated as a dashed line connecting a subset of the available hardware electrodes. In some embodiments, the subset includes a portion of the hardware electrodes. In some embodiments, the subset includes all of the hardware electrodes e.g. of an imaging tool e.g. of a volume in space.

In some embodiments, width of the hardware electrodes is w and a distance between hardware electrodes is distance d (in one dimension).

In some embodiments, a spatial resolution, SR is a function of a number of hardware electrodes (N) in a virtual electrode; and the width and distance between the hardware electrodes (W and d, respectively) where, in some embodiments:

$$S R = f ( \frac{N}{W d} ) \qquad Equation 1$$

Where a target volumetric accuracy in 3-D, V A(x,y,z)), where \tilde{x} is a number of dimensions is:

$$V A = \frac{{V}_{real}}{{V}_{image}} = f ( \tilde{x} ) \qquad Equation 2$$

FIG. 14 is a simplified schematic of a plurality of hardware electrodes being operated as a virtual electrode, according to some embodiments of the invention.

In some embodiments, hardware electrodes are illustrated as solid outlined circles.

FIG. 14 illustrates generation of a virtual electrode, according to some embodiments of the invention, where the virtual electrode is illustrated by circles outlined with dotted lines. In some embodiments, by introducing different delays (e.g. phase changes) to signals received A, B, C, D, a geometry of the virtual electrode is selected. Where the different delays are, in some embodiments, implemented by hardware and/or in some embodiments, implemented by software e.g. at a processor 1490.

In some embodiments, a constant increase in delay of the signal is introduced along a length of the virtual electrode, shifting an orientation of the virtual electrode, for example, as illustrated in FIG. 14.

In some embodiments, a shape and/or size of a virtual electrode is selected by controlling delays introduced. Similarly, in some embodiments, delays (e.g. phase changes) are introduced to transmitted signals to control a shape and/or size of a signal transmitting virtual electrode.

### Examples of Structure Imaging

FIG. 10 is a flow chart of a method of measurement of a structure using electrodes positioned on at least two sides of the structure, according to some embodiments of the invention.

At 1000, in some embodiments, a first electrode is positioned on a first side of a structure to be measured and a second electrode is positioned on the second side of the structure to be measured.

At 1002, in some embodiments, electrical signals are transmitted from and/or sensed at the electrodes to collect measurement data.

At 1004, in some embodiments, an image is generated using data collected from both sides of the structure. Alternatively or additionally, in some embodiments, a measurement is generated (e.g. thickness of the structure, in at least on dimension) using the data collected from both sides of the structure.

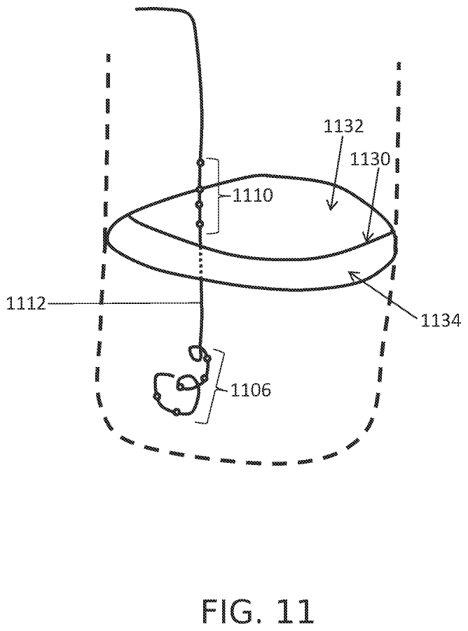

FIG. 11 is a simplified schematic of an imaging tool 1112 where a first set of electrodes 1106 are positioned on a first side of tissue to be imaged and a second set of electrodes 1110 are positioned on a second side of tissue to be imaged, according to some embodiments of the invention.

In some embodiments, the structure to be measured includes a valve, for example, a heart valve. Where, in some embodiments, a portion of imaging tool 1112 connecting first set 1106 and second set 1110 passes through a commissure 1130 of the valve. In some embodiments, the portion of the tool connecting the first and second sets of electrodes lacks electrodes. In some embodiments, the connecting portion is non-elastic e.g. rigid. Illustrated in FIG. 11 are a first and a second valve leaflet 1130, 1132 though it is to be understood that the measurement tool and/or method of measurement, in some embodiments, is used in valves with different numbers of leaflets, for example, more than two leaflets.

In some embodiments, an image of the valve annulus is generated from the collected data.

FIGS. 12A-B are simplified schematics cross sections showing placement of a valve clip 1242 assisted by an imaging tool 1212, according to some embodiments of the invention.

In some embodiments, the valve is a heart valve, for example, a mitral valve where volume 1244 is the left atrium. In some embodiments, a portion of imaging tool 1212 including a first set of electrodes 1206 is positioned in the left ventricle (not illustrated) and a portion of imaging tool 1212 including a second set of electrodes 1210 is positioned in left atrium 1244. In some embodiments, imaging tool 1212 is used to image the mitral valve, where the imaging is used in positioning of valve clip 1242.

FIG. 12A in some embodiments illustrates positioning of valve clip 1242 by a catheter 1246 and, in some embodiments, FIG. 12B illustrates positioning of the valve clip at leaflets 1240.

Examples of Inverse Problem Solutions and their Use

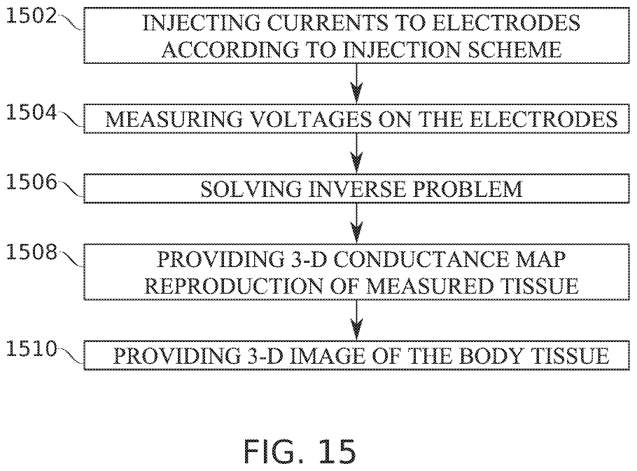

Reference is now made to FIG. 15, which is a flow chart depicting a method of dielectric mapping, optionally for imaging a body volume or for reconstructing body volume, according to some embodiments of the present disclosure.

The body volume may include or be a body tissue.

Currents may be injected at block 1502, in some embodiments, for example by control unit 114, to electrodes deployed on a patient's body, such as electrodes 108, and/or to intra-body electrodes, such as electrodes 106, according to an injection scheme (block 1502). Injection schemes may include a time/frequency transmission scheme. Injection schemes may be controlled and monitored by controller 114.

At block 1504, in some embodiments, voltages are measured on electrodes (e.g., on all electrodes) for example using a signal generator/measurer which is a component of and/or under control of control unit 114.

At block 1506, in some embodiments, an inverse problem (calculation and production of 3-D spatial distribution of conductances of body tissues based on the currents/voltages measured) may be solved, e.g. by control unit 114.

At block 1508, in some embodiments, a 3-D conductance map (3-D distribution of conductance measurements, also referred to herein as conductivity map) is optionally obtained and/or provided for display.

At block 1510, in some embodiments, a 3-D image of the body tissue is optionally produced and/or presented for display, based on the 3-D conductance map.

It will be appreciated that the method of FIG. 15 may include a precursor to block 1502 of placing the surface electrodes (if used) on a patient and of inserting the intrabody electrodes (if used) into the patient. However, in some embodiments, the method excludes any surgical steps. For example, the method comprises receiving data set values indicative of currents applied to the excitation electrodes (for example current values, electrode charge values, and/or electric field values at the electrode in question), and/or indicative of values indicative of voltage measured at the measurement electrodes (for example voltage values, impedance values, electric field values); and performing the disclosed data processing on the received data sets to generate a dielectric map and, optionally, an image based on the dielectric map.

The methods referred to above generically refer to “solving the inverse problem”, that is, to finding a spatial distribution of conductances (or other dielectric quantities) given spatially located field sources (resulting from injected currents) and spatially located field (voltage) measurements. Many different approaches to solving this problem are known, some of which involve a form of optimization to find a spatial distribution of conductances consistent with the field sources and measurements.

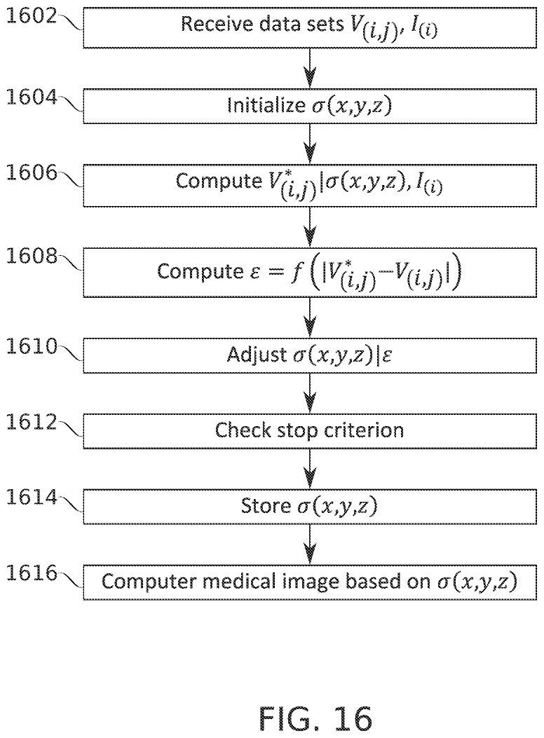

Reference is now made to FIG. 16, which is a flowchart schematically representing a method of finding a solution to the inverse problem, according to some embodiments of the present disclosure.

In overview, FIG. 16 describes a model of the spatial distribution of conductances σ(x,yz) which may be initialized to a starting guess, then optimized to be consistent with a set of current values I₍ᵢ₎, where i designates an electrode at a known position in a reference frame, and I is a value indicative of the current applied to that electrode, and a set of voltage values v_{(i,j)} is indicative of a measured voltage at electrode j of known position in the reference frame in response to current applied to electrode i. The current values I₍ᵢ₎, may be fixed parameters known in advance; for example set to a fixed value of magnitude and frequency of a current waveform, in which case I₍ᵢ₎ is applicable to all data sets v_{(i,j)}. Alternatively, I₍ᵢ₎, may vary; in which case respective values of I₍ᵢ₎, are included in the data set. The voltages and currents may be real-valued (for example if real-valued conductance is mapped) or may be complex-valued (for example if complex conductance or admittance is mapped).

The method, in some embodiments, comprises:
Receiving 1602 the collection V_{(i,j)} of a plurality of data sets v_{(i,j)} and I₍ᵢ₎.
Initializing 1604 an initial “guess” of σ(x,yz). The initial guess may be random or may be based on a previously calculated σ(x,y,z) calculated under related conditions, for example as described in more detail below.
Modeled values V_{(i,j)}* of measured voltages are calculated 1606 using physics knowledge: for example Maxwell's equations or Laplace equations, applied to the applied current values I₍ᵢ₎, (or simply I if fixed and predefined), the known positions of the electrodes i and j and the current σ(x,y,z), for example the initial guess on the first iteration.
In some embodiments, an error signal £ is computed 1608 as a function of the magnitude of the difference between measured and modeled voltage values. The function may be simple—for example the absolute or squared difference—or may include further terms to guide optimization. This may be, for example based on soft constraints as further detailed below, and/or for example based on the entropy of σ(x,y,z), as is well known in the art of function optimization.
The error signal is used to adjust 1610 σ(x,y,z) using gradient descent on a gradient of the error and/or using another well-known optimization technique (treating the parameters defining σ(x,y,z) as the optimization parameters to be optimized).
Before or after updating σ(x,y,z), the method involves, in some embodiments, checking 1612 whether a stopping criterion has been met: for example in terms of the error signal falling below a threshold value or changing by less than a threshold amount compared to the previous iteration(s).
If the stopping criterion is not met, the method circles back to computing 1606 modelled voltages.
Otherwise, the method stores 1614 σ(x,y,z) and either terminates or proceeds to optional processes, such as computing 1616 a medical image based on σ(x,y,z).

Numerous ways of defining σ(x,y,z) are envisaged. In one example, σ(x,y,z) is defined in terms of a linear superposition of base conductance distributions for a target organ to be mapped that have been derived before by other means; for example other optimization techniques, and/or based on other imaging modalities across a group of subjects. In this case, the optimization parameters are the superposition coefficients and optimization is based on numerically calculated gradients or other means, such as Monte Carlo methods.

In another example, σ(x,y,z) is defined on a mesh of conductances, and Finite Element Analysis (FEA) is used to calculate the forward model (V*). In some embodiments the mesh may be a uniform Cartesian mesh defined in terms of x, y and z axes. In some embodiments, a non-uniform mesh (e.g., a tetrahedron mesh) is used, adjusted based on the locations of the electrodes (and hence the location of the available information), as is well known in the field of FEA. Where measurements from multiple frames of reference are obtained, the mesh may be determined dynamically and optimized in each instance or, in embodiments that favor efficiency, a mesh may be predefined, for example based on catheter electrode configuration, for all frames of reference. Irrespective of how the mesh/cells of the FEA model are defined, in some embodiments the (e.g., tetrahedron) conductance values of the FEA model are the optimization parameters adjusted based on the error signal.

Difficulty in solving the optimization problem of finding σ(x,y,z) is potentially increased in that in order to achieve desirable levels of resolution, many parameters need to be adjusted based on data from an inevitably limited number of electrodes.

While various regularization approaches are known to help with this problem, the inventors have realized that it is possible to use known dielectric characteristics of a catheter or other tool placed in the region to be mapped to constrain the optimization. This approach is applicable irrespective of the identity of the electrodes used for field generation and measurement. It may, for example, be applied to embodiments in which only surface electrodes are used for both measurement and field generation. In these case, the catheter is placed in the region merely to provide constraint data without participating in the measurement. Evidently, in other embodiments in which intrabody electrodes participate in field generation or measurement, the catheter may have a dual function of carrying the intrabody electrodes and providing constraint data. In some embodiments, constraint elements not on the catheter carrying intrabody electrodes may be used; for example, dielectric and/or conductive parts on other tools disposed in the body, conductive and/or dielectric markers permanently or temporarily secured to the body or organ, and so forth.

Known information about the catheter (or other known body) may take various forms, for example: a distribution of the dielectric properties of the catheter, such a distribution combined with a known position of the catheter in an external reference frame (for example defined by the surface electrodes); a length and known dielectric properties of a plastic part of the catheter; a position and/or configuration of electrodes on the catheter; a distance between electrode pairs on the catheter; the position of metal elements such as electrodes on the catheter that are or are not used for field generation or measurement; and the like. These and other items of information about the catheter will potentially be most informative when available in the same reference frame as the measurements. For example, this would be the case for measurements made with the surface electrodes, where the position of the catheter is known within the reference frame of the surface electrodes fixed to the body. Position detection of the catheter may be by external means, such as medical imaging, for example computer tomography or magnetic resonance imaging, or as described further below. This would also be the case where measurements are taken in the reference frame of the catheter itself that is where the emitting and measuring electrodes are both disposed on the catheter, and the constraints are defined on the catheter, as well. However, some measurements such as distance measurements between landmarks such as electrodes on the catheter are invariant to the frame of reference and such constraints can be used irrespective of the frame of reference, by detecting the landmarks in the current iteration of σ(x,y,z) and using this to constrain the optimization.

The constraints may be used to influence the optimization discussed above as soft or hard constraints, as is known in the art. A soft constraint is provided by adding an additional term punishing deviations from the constraint to the function defining the error signal computed at step 1608, so that the resulting gradients (in the case of gradient descent) are biased towards solutions that are consistent with the constraint. For example, where a distribution of dielectric properties is known in the frame of reference of reconstruction, such as when all electrodes are provided on the catheter and the distribution of the dielectric properties of the catheter are used as constraint, the function defining the error signal may comprise a term penalizing the magnitude of deviation of σ(x,y,z) from the known dielectric distribution in the region of the catheter, averaged over the catheter. In addition or alternatively, for example, the function may comprise a term penalizing a deviation from the know distance between electrodes detected as landmarks in σ(x,y,z), or between other landmarks. Implemented as hard constraints, the adjustment at step 1610, in some embodiment, is altered to include an additional adjustment in addition to the optimization update. The additional adjustment ensures that after step 1614 σ(x,y,z) meets the constraint and may, for example, include, in the region where constraints are defined in terms of a dielectric distribution, setting values of σ(x,y,z) to that dielectric distribution, or scaling, rotating or otherwise transforming σ(x,y,z) to be consistent with distance-based constraints, as the case may be.

In some embodiments, measurements are made and fields generated with moving intrabody electrodes. For example, the electrodes may be disposed on a moving catheter or other tool. As the intrabody electrodes move from location to location, respective reference frame of measurements and corresponding spatial distributions are generated. The electrodes used for the measurements and corresponding field generation may be only on the catheter or include electrodes disposed in a fixed relationship to the body (fixed electrodes), such as described above. For combining information from fixed and moving electrode, the locations of the fixed electrodes may be transformed into a common moving frame of reference common with the intrabody electrodes and moving with the catheter. In either case, a sequence of dielectric maps (or frames) is generated corresponding to locations through which the catheter travels. These maps are, in some embodiments, combined to obtain combined map of the region of interest through which the catheter travels.

### General

It is expected that during the life of a patent maturing from this application many relevant imaging technologies and/or imaging tools will be developed; the scope of the term “imaging” and “imaging tool(s)” is intended to include all such new technologies a priori.

As used herein with reference to quantity or value, the term “about” means “within ±20% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

In addition, any priority document(s) of this application is/are hereby incorporated herein by reference in its/their entirety.

1. A tissue structure imaging method comprising:
receiving a first set and a second set of electrical field measurement data measured by respective first and second in-body electrodes from positions on respective first and second sides of the tissue structure; and
generating an image of said tissue structure using said first and second sets of electrical field measurement data;
wherein the positions on the first side and the second side are spatial locations with the tissue structure between them, and wherein there is, for each spatial location, a surface of the tissue exposed to it across a fluid medium, and the exposed surfaces each have a non-overlapping portion comprising at least 20% of their surface.

2. The method of claim 1, wherein the generating the image comprises transforming measurements to locations under a constraint that the two sets of electrical field measurements are of different sides of the same tissue structure.

3. The method of claim 1, wherein the exposed surfaces are on lines of sight from each of the positions on the first side and the second side.

4. The method of claim 3, wherein the lines of sight meet at 180°.

5. The method of claim 1, wherein each line of sight defines a straight path including the spatial location and the tissue structure, and passing through a medium without the straight path penetrating through or into a solid structure other than the tissue structure.

6. The method of claim 1, wherein the measurements collected from both sides of the tissue structure comprise measurements indicative of currents applied to the first electrode and voltage measurements by the second electrode; and measurements indicative of currents applied to the second electrode and voltage measurements by the first electrode.

7. The method of claim 1, wherein the generating comprises solving the inverse problem to produce an image of the tissue structure using the measurement data, including comparing differences in measurement data obtained from the positions on the first and second sides to constrain the solution of the inverse problem.

8. The method of claim 1, wherein a respective straight path from each of the first and second electrodes to the tissue structure passes through a fluid medium without the straight path penetrating through or into a solid structure, and wherein the generating comprises using this positioning as a constraint on the solution of the inverse problem.

9. The method of claim 1, wherein the generating the image comprises generating a respective first location cloud and second location cloud for locations of portions of the tissue structure using the first and second sets of electrical field measurement data, and then combining the first location cloud and second location cloud to generate the image.

10. The method of claim 9, wherein the combining comprises averaging locations of corresponding features within the first and second location clouds.

11. The method of claim 9, wherein the combining comprises discarding at least one feature present in one of the first location cloud and the second location cloud, but not shown in the other.

12. The method of claim 1, comprising:
positioning the first in-body electrode on a first side of the tissue structure and the second in-body electrode on a second side of the tissue structure; and
using said electrodes to collect measurement data of one or more electrical fields passing through said tissue structure, the measurement data comprising the first set and the second set of electrical field measurement data.

13. The tissue structure imaging method according to claim 12, comprising positioning a third electrode on a third side of said tissue structure.

14. The tissue structure imaging method according claim 12, wherein said first electrode and said second electrode are both part of a same imaging tool.

15. The tissue structure imaging method according to claim 12, wherein said positioning includes positioning a first set of electrodes on said first side and a second set of electrodes on said second side.

16. The tissue structure imaging method according to claim 15, wherein said first set of electrodes and said second set of electrodes are both parts of a same imaging tool.

17. The tissue structure imaging method according to claim 12, wherein said positioning comprises inserting a portion of a tool with the first electrode through the tissue structure.

18. The tissue structure imaging method according to claim 12, wherein the tissue structure is a vascular system valve.

19. The tissue structure imaging method according to claim 12, wherein the tissue structure is a heart valve.

20. The tissue structure imaging method according to claim 12, comprising using said image to guiding placement of a device with respect to the tissue structure.

21. The tissue structure imaging method according to claim 20, wherein the tissue structure is a cardiovascular valve and said device is a valve clip.

22. The tissue structure imaging method according to claim 21, wherein said valve clip comprises one or more of said electrodes.

23. The tissue structure imaging method according to claim 21, wherein a delivery device for said valve clip comprises one or more of said electrodes.

24. The tissue structure imaging method according to claim 12, comprising generating an electrical field at a volume including a volume of the tissue structure; wherein said measurement data comprises features of said electrical field affected by the tissue structure.

25. The tissue structure imaging method according to claim 24, wherein said generating an electrical field comprises generating at least three electromagnetic fields, where said fields have crossing orientations.

26. The tissue structure imaging method according to claim 25, wherein said fields have different frequencies.

27-51. (canceled)

52. A tissue imaging method of imaging a target tissue comprising:
providing a plurality of hardware electrodes;
sending and receiving electrical signals using said plurality of hardware electrodes to produce measurement data;
selecting a virtual electrode model, where each said virtual electrode includes two or more hardware electrodes;
processing said measurement data using said virtual electrode model to provide a processed data output; and
reconstructing an image using said processed data output.

53-56. (canceled)
